SEQUENCE LISTING (22 sequences)

SEQ ID NO: 1 - protein, 33 aa, Mus musculus
PIPITDLADNIERLKANDGKLFSQEYESIDPGQ

SEQ ID NO: 2 - protein, 33 aa, Rat coronavirus; identical to SEQ ID NO: 1

SEQ ID NO: 3 - protein, 33 aa, Homo sapiens; identical to SEQ ID NO: 1

SEQ ID NO: 4 - protein, 33 aa, Mus musculus
PIPITDMAEHMERLKANDSLKLSQEYESIDPGQ

SEQ ID NO: 5 - protein, 33 aa, Rat coronavirus; identical to SEQ ID NO: 4

SEQ ID NO: 6 - protein, 33 aa, Homo sapiens
PIPIADMAEHTERLKANDSLKLSQEYESIDPGQ

SEQ ID NO: 7 - protein, 33 aa, Mus musculus
PIPILELADHIERLKANDNLKFSQEYESIDPGQ

SEQ ID NO: 8 - protein, 33 aa, Homo sapiens; identical to SEQ ID NO: 7

SEQ ID NO: 9 - protein, 24 aa, Homo sapiens
DMAEHTERLKANDSLKLSQEYESI

SEQ ID NO: 10 - protein, 24 aa, Rattus rattus
DLADNIERLKANDGLKFSQEYESI

SEQ ID NO: 11 - protein, 24 aa, Rattus rattus
ELADHIERLKANDNLKFSQEYESI

SEQ ID NO: 12 - protein, 24 aa, Rattus rattus
KLEEEINRRMADDNKIFREEFNAL

SEQ ID NO: 13 - protein, 10 aa, Homo sapiens
DMAEHTERLK

SEQ ID NO: 14 - protein, 4 aa, Homo sapiens
ANDS

SEQ ID NO: 15 - protein, 10 aa, Homo sapiens
LKLSQEYESI

SEQ ID NO: 16 - protein, 14 aa, Artificial Sequence
  Synthetic Construct
  MISC_FEATURE (3)..(3): Xaa is Thr or Met
EHXERLKANDSLKL

SEQ ID NO: 17 - protein, 35 aa, Homo sapiens
GRKKRRQRRRCDMAEHTERLKANDSLKLSQEYESI

SEQ ID NO: 18 - protein, 35 aa, Homo sapiens
GRKKRRQRRRCDLADNIERLKANDGLKFSQEYESI

SEQ ID NO: 19 - protein, 35 aa, Homo sapiens
GRKKRRQRRRCELADHIERLKANDNLKFSQEYESI

SEQ ID NO: 20 - protein, 35 aa, Homo sapiens
GRKKRRQRRRCKLEEEINRRMADDNKIFREEFNAL

SEQ ID NO: 21 - protein, 21 aa, Homo sapiens
GRKKRRQRRRCDMAEHTERLK

SEQ ID NO: 22 - protein, 25 aa, Homo sapiens
  MISC_FEATURE (14)..(14): Xaa is Thr or Met
GRKKRRQRRRCEHXERLKANDSLKL

(19) United States
(12) Patent Application Publication
(10) Pub. No.: US 2020/0093890 A1
(43) Pub. Date: Mar. 26, 2020

(54) COMPOSITIONS AND METHODS FOR TREATING ALZHEIMER'S DISEASE

(71) Applicants: CASE WESTERN RESERVE UNIVERSITY, Cleveland, OH (US); OHIO STATE UNIVERSITY, Columbus, OH (US)

(72) Inventors: Yingjie Shen, Cleveland, OH (US); Yuanzheng Gu, Columbus, OH (US); Jerry Silver, Cleveland, OH (US); Bradley Lang, Cleveland, OH (US)

(21) Appl. No.: 16/619,317

(22) PCT Filed: Jun. 5, 2018
(86) PCT No.: PCT/US2018/036116

§ 371 Date: Dec. 4, 2019

Related U.S. Application Data
(60) Provisional application No. 62/515,272, filed on Jun. 5, 2017.

(51) Int. Cl. A61K 38/17; A61P 25/28; A61K 38/16; A61K 45/06

(52) U.S. Cl. CPC A61K 38/1716; A61K 45/06; A61K 38/162; A61P 25/28

(57) ABSTRACT

A method inhibiting and/or reducing β-amyloid accumulation and/or Tau aggregation in a subject in need thereof includes administering to the subject a therapeutic agent that inhibits one or more of catalytic activity, signaling, and function of the LAR family phosphatases.

### RELATED APPLICATION

This application claims priority from U.S. Provisional Application No. 62/515,272, filed Jun. 5, 2017, the subject matter of which is incorporated herein by reference in its entirety.

### TECHNICAL FIELD

This application relates to compositions and methods for inhibiting or reducing the activity, signaling, and/or function of leukocyte-common antigen related (LAR) family of phosphatases and to methods and compositions for inhibiting β-amyloidosis and treating Alzheimer's disease.

### BACKGROUND

A definitive pathological hallmark of Alzheimer's disease (AD) is the progressive aggregation of β-amyloid (Aβ) peptides in the brain, a process also known as β-amyloidosis, which is often accompanied by neuroinflammation and formation of neurofibrillary tangles containing Tau, a microtubule binding protein.

Although the etiological mechanisms of AD have been an ongoing debate, concrete evidence from human genetic studies showed that overproduction of Aβ due to gene mutations inevitably inflicts cascades of cytotoxic events, ultimately leading to neurodegeneration and decay of brain functions. Accumulation of Aβ peptides, especially in their soluble forms, is therefore recognized as a key culprit in the development of AD. In the brain, Aβ peptides mainly derive from sequential cleavage of neuronal amyloid precursor protein (APP) by the β- and γ-secretases. However, despite decades of research, molecular regulation of the amyloidogenic secretase activities remains poorly understood, hindering the design of therapeutics to specifically target the APP amyloidogenic pathway.

Pharmacological inhibition of the β- and γ-secretase activities, although effective in suppressing Aβ production, interferes with physiological function of the secretases on their other substrates. Such intervention strategies therefore are often innately associated with untoward side effects, which have led to several failed clinical trials in the past. To date, no therapeutic regimen is available to prevent the onset of AD or curtail its progression.

### SUMMARY

Embodiments described herein relate to methods of inhibiting and/or reducing β-amyloid accumulation and/or Tau aggregation in a subject in need thereof. The methods include administering to the subject a therapeutic agent that inhibits one or more of catalytic activity, signaling, and function of the LAR family phosphatases.

In some embodiments, the LAR family phosphatase is a receptor protein tyrosine phosphatase sigma (PTPσ), and the therapeutic agent includes a therapeutic peptide having an amino acid sequence that is at least about 65%, at least about 70%, at least about 75%, at least about 80%, at least about 85%, at least about 90%, or at least about 95% identical to about 10 to about 20 consecutive amino acids of the wedge domain of PTPσ. For example, therapeutic agent can include a therapeutic peptide selected from the group consisting of SEQ ID NOs: 9-13 and 16.

In other embodiments, the LAR family phosphatase is a receptor protein tyrosine phosphatase sigma (PTPσ), and the therapeutic agent can include a therapeutic peptide at least about 65%, at least about 70%, at least about 75%, at least about 80%, at least about 85%, at least about 90%, or at least about 95% identical to the amino acid sequence of SEQ ID NO: 16. The therapeutic peptide can include, for example, a conservative substitution of an amino acid of at least one, two, three, or four of residue 4, 5, 6, 7, 9, 10, 12, or 13 of SEQ ID NO: 16.

In some embodiments, the therapeutic agent is administered systemically or locally to the subject or to a neural cell, glial cell, glial progenitor cell, or a neural progenitor cell.

In other embodiments, the therapeutic agent includes a transport moiety that is linked to the therapeutic peptide and facilitates uptake of the therapeutic peptide by the cell. For example, the transport moiety can be an HIV Tat transport moiety.

In still other embodiments, the cell is in a subject being treated, and the therapeutic agent is administered locally or systemically to the subject being treated.

In yet other embodiments, the therapeutic peptide is expressed by a cell of the subject.

Embodiments herein also relate to methods of treating diseases, disorders, and/or conditions associated with β-amyloid accumulation and/or Tau aggregation in a subject in need thereof. The methods include administering to the subject a therapeutic agent that inhibits one or more of catalytic activity, signaling, and function of the LAR family phosphatases.

In some embodiments, the disease, disorder, and/or condition, includes at least one of a disease, disorder, and/or condition of the nervous system.

In other embodiments, the disease, disorder, and/or condition of the nervous system includes at least one of a neurological disorder, neuropsychiatric disorder, neural injury, neural toxicity disorder, a neuropathic pain, and neural degenerative disorders.

For example, the neurological disorder can include at least one of Alzheimer's disease or dementias related to Alzheimer's disease.

### BRIEF DESCRIPTION OF THE DRAWINGS

FIGS. 1(A-I) illustrate images and immunoblots showing PTPσ is an APP binding partner in the brain. A-F, Colocalization of PTPσ (A) and APP (B) in hippocampal CA1 neurons of adult rat is shown by confocal imaging. Nuclei of CA1 neurons are stained with DAPI (C). D, Merge of three channels. Scale bar, 50 μm. E, Zoom-in image of the soma layer in D. Arrows, intensive colocalization of PTPσ and APP in the initial segments of apical dendrites; arrow heads, punctates of colocalization in the perinuclear regions. Scale bar, 20 μm. F, Zoom-in image of the very fine grained punctates in the axonal compartment in D. Arrows points to the colocalization of PTPσ and APP in axons projecting perpendicular to the focal plane. Scale bar, 10 μm. G, Schematic diagram of PTPσ expressed on cell surface as a two-subunit complex. PTPσ is post-translationally processed into an extracellular domain (ECD) and a transmembrane-intracellular domain (ICD). These two subunits associate with each other through noncovalent bond. Ig-like, immunoglobulin-like domains; FNIII-like, fibronectin III-like domains; D1 and D2, two phosphatase domains. H, I, Co-immunoprecipitation (co-IP) of PTPσ and APP from mouse forebrain lysates. Left panels, expression of PTPσ and APP in mouse forebrains. Right panels, IP using an antibody specific for the C-terminus (C-term) of APP. Full length APP (APP FL) is detected by anti-APP C-term antibody. H, PTPσ co-IP with APP from forebrain lysates of wild type but not PTPσ-deficient mice (Balb/c background), detected by an antibody against PTPσ-ECD. I, PTPG co-IP with APP from forebrain lysates of wild type but not APP knockout mice (B6 background), detected by an antibody against PTPσ-ICD. Dotted lines in I indicate lanes on the same western blot exposure that were moved adjacent to each other. Images shown are representatives of at least three independent experiments.

FIGS. 2(A-I) illustrate a schematic diagram, immunoblots, and graphs showing genetic depletion of PTPG reduces β-amyloidogenic products of APP. A, Schematic diagram showing amyloidogenic processing of APP by the β- and γ-secretases. Full length APP (APP FL) is cleaved by β-secretase into soluble N-terminal (sAPPβ) and C-terminal (CTFβ) fragments. APP CTFβ can be further processed by γ-secretase into a C-terminal intracellular domain (AICD) and an Aβ peptide. Aggregation of Aβ is a definitive pathology hallmark of AD. B, PTPG deficiency reduces the level of an APP CTF at about 15 KD in mouse forebrain lysates, without affecting the expression of APP FL. Antibody against the C-terminus of APP recognizes APP FL and CTFs of both mouse and human origins. C and D, The 15 KD APP CTF is identified as CTFβ by immunoprecipitation (IP) followed with western blot analysis, using a pair of antibodies as marked in the diagram (A). Antibodies against amino acids 1-16 of Aβ (anti-Aβ1-16) detect CTFβ but not CTFα, as the epitope is absent in CTFα. C, Mouse endogenous CTFβ level is reduced in PTPσ-deficient mouse brains. 4 repeated experiments were quantified by densitometry. D, Human transgenic CTFβ level is reduced in PTPσ-deficient mouse brains harboring human APP-SwDI transgene. 6 repeated experiments were quantified by densitometry. Within each experiment in both C and D, the value from PTPG deficient sample was normalized to that from the sample with wild type PTPσ. E and F, PTPG deficiency reduces the levels of Aβ40 (E) and Aβ42 (F) in TgAPP-SwDI mice as measured by ELISA assays. n=12 for each group. The mean values from PTPG deficient samples was normalized to that from the samples with wild type PTPσ. G and H, Aβ deposition in the hippocampus of 10-month old TgAPP-SwDI mice. Aβ (green) is detected by immunofluorescent staining using anti-A antibodies clone 6E10 (G) and clone 4G8 (H). DAPI staining is shown. PTPG deficiency significantly decreases Aβ burden in the brains of TgAPP-SwDI mice. H, Upper panels, the stratum oriens layer between dorsal subiculum (DS) and CA1 (also shown with arrows in G); middle panels, oriens layer between CA1 and CA2; lower panels, the hilus of dentate gyrus (DG, also shown with arrow heads in g). Left column, control staining without primary antibody (no 10° Ab). No Aβ signal is detected in non-transgenic mice (data not shown). Scale bars, 500 μm in G and 100 μm in h. Images shown are a representative pair among 5 pairs of age- and sex-matched mice of 9- to 11-month old. I, Genetic depletion of PTPσ suppresses the progression of Aβ pathology in TgAPP-SwDI mice. ImageJ quantification of Aβ immunofluorescent staining (with 6E10) in DG hilus from 9- and 16-month old TgAPP-SwDI mice. n=3 for each group. Total integrated density of Aβ in DG hilus was normalized to the area size of the hilus to yield the average intensity as show in the bar graph. Mean value of each group was normalized to that of 16 month old TgAPP-SwDI mice expressing wild type PTPσ. All p values, Student's t test, 2-tailed. Error bars, SEM.

FIGS. 3(A-C) illustrate an immunoblot, graph, and plot showing lower affinity between BACE1 and APP in PTPσ-deficient brains. A, Coimmunoprecipitation experiments show nearly equal BACE1-APP association in wild type and PTPσ-deficient mouse brains under mild detergent condition (1% NP40). However, in PTPσ-deficient brains, BACE1-APP association detected by co-immunoprecipitation is more vulnerable to increased detergent stringency as compared to that in wild type brains. Panels of blots show full length APP (APP FL) pulled down with an anti-BACE1 antibody from mouse forebrain lysates. NP40, Nonidet P-40, non-ionic detergent. SDS, Sodium dodecyl sulfate, ionic detergent. B, Co-immunoprecipitation under buffer condition with 1% NP40 and 0.3% SDS, as shown in the middle panel of A, were repeated three times. Each experiment was quantified by densitometry, and the value from PTPσ-deficient sample was calculated as a percentage of that from the wild type sample (also shown as orange points in C). Error bar, SEM. p value, Student's t test, 2-tailed. C, Co-immunoprecipitation experiments were repeated under each detergent condition. The percentage values shown in dots are derived using the same method as in B. Bars represent means. Increasingly stringent buffer conditions manifest a lower BACE1-APP affinity in PTPσ-deficient brains. p value and R2, linear regression.

FIGS. 4(A-F) illustrate immunoblots showing PTPσ does not generically modulate β and γ secretases. Neither expression levels of the secretases or their activities on other major substrates are affected by PTPσ depletion. Mouse forebrain lysates with or without PTPσ were analyzed by western blot. A and B, PTPσ deficiency does not change expression level of BACE1 (A) or γ-secretase subunits (B). Presenilinl and 2 (PS1/2) are the catalytic subunits of γ-secretase, which are processed into Nterminal and C-terminal fragments (NTF and CTF) in their mature forms. Nicastrin, Presenilin Enhancer 2 (PEN2), and APH1 are other essential subunits of γ-secretase. C, PTPσ deficiency does not change the level of Neuregulin1 (NGR1) CTFβ, the C-terminal cleavage product by BACE1. NRG1 FL, full length Neuregulin1. D, The level of Notch cleavage product by γ-secretase is not affected by PTPσ deficiency. TMIC, Notch transmembrane/intracellular fragment, which can be cleaved by γ-secretase into a C-terminal intracellular domain NICD (detected by an antibody against Notch C-terminus in the upper panel, and by an antibody specific for γ-secretase cleaved NICD in the lower panel). E, Actin loading control for A and C. F, Actin loading control for B and D. All images shown are representatives of at least three independent experiments. All images shown are representatives of at least three independent experiments.

FIGS. 5(A-I) illustrate images and a graph showing PTPσ deficiency attenuates reactive astrogliosis in APP transgenic mice. Expression level of GFAP, a marker of reactive astrocytes, is suppressed in the brains of TgAPP-SwDI mice by PTPσ depletion. Representative images show GFAP and DAPI staining of nuclei in the brains of 9-month old TgAPP-SwDI mice. A-D, Dentate gyrus (DG) of the hippocampus; scale bars, 100 μm. E-H, Primary somatosensory cortex; scale bars, 200 μm. I, ImageJ quantification of GFAP level in DG hilus from TgAPP-SwDI mice aged between 9 to 11 months. APP-SwDI(−)PTPσ(+/+), non-transgenic wild type littermates (expressing PTPσ but not the human APP transgene). Total integrated density of GFAP in DG hilus was normalized to the area size of the hilus to yield average intensity as shown in the bar graph. Mean value of each group was normalized to that of APP-SwDI(−)PTPσ(+/+) mice (image not shown). APP-SwDI(−)PTPσ(+/+), n=4; APPSwDI(+) PTPσ(+/+), n=4; APP-SwDI(+)PTPσ(−/−), n=6. All p values, Student's t test, 2-tailed. Error bars, SEM.

FIGS. 6(A-G) illustrates images and a graph showing PTPσ deficiency protects APP transgenic mice from synaptic loss. Representative images show immunofluorescent staining of presynaptic marker Synaptophysin in the mossy fiber terminal zone of CA3 region. A-F, Synaptophysin, red; DAPI, blue. Scale bars, 100 μm. G, ImageJ quantification of Synaptophysin expression level in CA3 mossy fiber terminal zone from mice aged between 9 to 11 months. Total integrated density of Synaptophysin in CA3 mossy fiber terminal zone was normalized to the area size to yield average intensity as shown in the bar graph. Mean value of each group was normalized to that of wild type APP-SwDI(-)PTPσ(+/+) mice. APPSwDI(−)PTPσ(+/+), n=4; APP-SwDI(+)PTPσ(+/+), n=6; APP-SwDI(+)PTPσ(−/−), n=6. All p values, Student's t test, 2-tailed. Error bars, SEM.

FIGS. 7(A-H) illustrates a schematic diagram, images, and graph showing PTPσ deficiency mitigates Tau pathology in TgAPP-SwDI mice. A, Schematic diagram depicting distribution pattern of Tau aggregation detected by immunofluorescent staining using an anti-Tau antibody, in brains of 9 to 11 month-old TgAPP-SwDI transgenic mice. Aggregated Tau is found most prominently in the molecular layer of piriform and entorhinal cortex, and occasionally in hippocampal regions in APPSwDI(+)PTPσ(+/+) mice. B, PTPσ deficiency diminishes Tau aggregation. Bar graph shows quantification of Tau aggregation in coronal brain sections from 4 pairs of age- and sexmatched APP-SwDI(+)PTPσ(+/+) and APP-SwDI(+)PTPσ(−/−) mice of 9 to 11 month-old. For each pair, the value from APP-SwDI(+)PTPσ(−/−) sample is normalized to the value from APP-SwDI(+)PTPσ(+/+) sample. p value, Student's t test, 2-tailed. Error bar, SEM. C, D, Representative images of many areas with Tau aggregation in APP-SwDI(+)PTPσ(+/+) brains. F, G, Representative images of a few areas with Tau aggregation in age-matched APPSwDI(+)PTPσ(−/−) brains. C and F, Hippocampal regions. D-H, Piriform cortex. E, Staining of a section adjacent to d, but without primary antibody (no 10° Ab). H, no Tau aggregates are detected in aged-matched non-transgenic wild type littermates (expressing PTPσ but not the human APP transgene). Arrows points to Tau aggregates. Scale bars, 50 μm.

FIGS. 8(A-C) illustrate graphs showing PTPσ deficiency rescues behavioral deficits in TgAPP-SwDI mice. A, In the Y-maze assay, performance of spatial navigation is scored by the percentage of spontaneous alternations among total arm entries. Values are normalized to that of non-transgenic wild type APP-SwDI(−)PTPσ(+/+) mice within the colony. Compared to non-transgenic wild type mice, APP-SwDI(+)PTPσ(+/+) mice show deficit of short term spatial memory, which is rescued by genetic depletion of PTPσ in APP-SwDI(+)PTPσ(−/−) mice. APP-SwDI(−)PTPσ(+/+), n=23 (18 females and 5 males); APP-SwDI(+)PTPσ(+/+), n=52 (30 females and 22 males); APP-SwDI(+)PTPσ(−/−), n=35 (22 females and 13 males). Ages of all genotype groups are similarly distributed between 4 and 11 months. B, C, Novel object test. NO, novel object. FO, familiar object. Attention to NO is measured by the ratio of NO exploration to total object exploration (NO+FO) in terms of exploration time (B) and visiting frequency (C). Values are normalized to that of non-transgenic wild type mice. APP-SwDI(+)PTPσ(+/+) mice showed decreased interest in NO compared to wild type APPSwDI(−)PTPσ(+/+) mice. The deficit is reversed by PTPσ depletion in APP-SwDI(+)PTPσ(−/−) mice. APPSwDI(−)PTPσ(+/+), n=28 (19 females and 9 males); APP-SwDI(+)PTPσ(+/+), n=46 (32 females and 14 males); APP-SwDI(+)PTPσ(−/−), n=29 (21 females and 8 males). Ages of all groups are similarly distributed between 4 and 11 months. All p values, Student's t test, 2-tailed. Error bars, SEM.

FIG. 9 illustrates a graph showing PTPσ deficiency restores short-term spatial memory in TgAPP-SwDI mice. In the Y-maze assay, performance of spatial navigation is scored by the percentage of spontaneous alternations among total arm entries. The raw values shown here are before normalization in FIG. 6A. Compared to non-transgenic wild type APP-SwDI(−)PTPσ(+/+)mice, APP-SwDI(+)PTPσ(+/+) mice show deficit of short-term spatial memory, which is rescued by genetic depletion of PTPσ. APPSwDI(−)PTPσ(+/+), n=23 (18 females and 5 males); APP-SwDI(+)PTPσ(+/+), n=52 (30 females and 22 males); APP-SwDI(+)PTPσ(−/−), n=35 (22 females and 13 males). Ages of all genotype groups are similarly distributed between 4 and 11 months. All p values, Student's t test, 2-tailed. Error bars, SEM.

FIGS. 10(A-D) illustrate graphs showing PTPσ deficiency enhances novelty exploration by TgAPP-SwDI mice. NO, novel object. FO, familiar object. A and B, in novel object test, NO preference is measured by the ratio between NO and FO exploration, where NO/FO >1 indicates preference for NO. C and D, attention to NO is additionally measured by the discrimination index, NO/(NO+FO), the ratio of NO exploration to total object exploration (NO+FO). The raw values shown here in c and d are before normalization in FIGS. 6B and C. Mice of this colony show a low baseline of the NO/(NO+FO) discrimination index, likely inherited from their parental Balb/c line. For non-transgenic wild type APP-SwDI(−)PTPσ(+/+) mice, the discrimination index is slightly above 0.5 (chance value), similar to what was previously reported for the Balb/c wild type mice 27. Thus, a sole measurement of the discrimination index may not reveal the preference for NO as does the NO/FO ratio. Although not as sensitive in measuring object preference, the NO/(NO+FO) index is most commonly used as it provides a normalization of the NO exploration to total object exploration activity. While each has its own advantage and shortcoming, both NO/FO and NO/NO+FO measurements consistently show that the expression of TgAPP-SwDI gene leads to a deficit in attention to the NO, whereas genetic depletion of PTPσ restores novelty exploration to a level close to that of non-transgenic wild type mice. A and C, measurements in terms of exploration time. B and D, measurements in terms of visiting frequency. APP-SwDI(−)PTPσ(+/+), n=28 (19 females and 9 males); APP-SwDI(+)PTPσ(+/+), n=46 (32 females and 14 males); APP-SwDI(+)PTPσ(−/−), n=29 (21 females and 8 males). Ages of all groups are similarly distributed between 4 and 11 months. All p values, Student's t test, 2-tailed. Error bars, SEM.

FIGS. 11(A-C) illustrate PTPσ deficiency improves behavioral performance of TgAPP-SwInd mice. A, Performance of spatial navigation is scored by the percentage of spontaneous alternations among total arm entries in the Y-maze assay. Compared to APPSwInd(+)PTPσ(+/+) mice, APPSwInd(+)PTPσ(−/−) mice showed improved short-term spatial memory. APPSwInd(+)PTPσ(+/+), n=40 (20 females and 20 males); APP-SwInd(+)PTPσ(−/−), n=18 (9 females and 9 males). Ages of both genotype groups are similarly distributed between 4 and 11 months. B, C, Novel object test. NO, novel object. FO, familiar object. NO preference is measured by the ratio of NO exploration time to total object exploration time (B) and the ratio of NO exploration time to FO exploration time (C). PTPσ depletion significantly improves novelty preference in these transgenic mice. APPSwInd(+)PTPσ(+/+), n=43 (21 females and 22 males); APP-SwInd(+)PTPσ(−/−), n=24 (10 females and 14 males). Ages of both groups are similarly distributed between 5 and 15 months. All p values, Student's t test, 2-tailed. Error bars, SEM.

FIG. 12 illustrates an immunoblot showing the effects ISP in combination with a γ-secretase inhibitor on APP processing compared to a γ-secretase inhibitor administered alone or a BACE1 inhibitor administered in combination with a γ-secretase inhibitor.

### DETAILED DESCRIPTION

The embodiments described herein are not limited to the particular methodology, protocols, and reagents, etc., and as such may vary. The terminology used herein is for the purpose of describing particular embodiments only, and is not intended to limit the scope of the present invention, which is defined solely by the claims. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and does not constitute any admission as to the correctness of the dates or contents of these documents.

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art.

As used herein, “one or more of a, b, and c” means a, b, c, ab, ac, bc, or abc. The use of “or” herein is the inclusive or.

As used herein, the term “administering” to a patient includes dispensing, delivering or applying an active compound in a pharmaceutical formulation to a subject by any suitable route for delivery of the active compound to the desired location in the subject (e.g., to thereby contact a desired cell such as a desired neuron), including administration into the cerebrospinal fluid or across the blood-brain barrier, delivery by either the parenteral or oral route, intramuscular injection, subcutaneous or intradermal injection, intravenous injection, buccal administration, transdermal delivery and administration by the rectal, colonic, vaginal, intranasal or respiratory tract route. The agents may, for example, be administered to a comatose, anesthetized or paralyzed subject via an intravenous injection or may be administered intravenously to a pregnant subject to stimulate axonal growth in a fetus. Specific routes of administration may include topical application (such as by eyedrops, creams or erodible formulations to be placed under the eyelid), intraocular injection into the aqueous or the vitreous humor, injection into the external layers of the eye, such as via subconjunctival injection or subtenon injection, parenteral administration or via oral routes.

As used herein, the term “antibody”, includes human and animal mAbs, and preparations of polyclonal antibodies, synthetic antibodies, including recombinant antibodies (antisera), chimeric antibodies, including humanized antibodies, anti-idiotopic antibodies and derivatives thereof. A portion or fragment of an antibody refers to a region of an antibody that retains at least part of its ability (binding specificity and affinity) to bind to a specified epitope. The term “epitope” or “antigenic determinant” refers to a site on an antigen to which antibody paratope binds. Epitopes formed from contiguous amino acids are typically retained on exposure to denaturing solvents whereas epitopes formed by tertiary folding are typically lost on treatment with denaturing solvents. An epitope typically includes at least 3, at least 5, or 8 to 10, or about 13 to 15 amino acids in a unique spatial conformation. Methods of determining spatial conformation of epitopes include, for example, x-ray crystallography and 2-dimensional nuclear magnetic resonance. See, e.g., 66 EPITOPE MAPPING PROTOCOLS IN METS. IN MOLECULAR BIO. (Morris, ed., 1996); Burke et al., 170 J. Inf. Dis. 1110-19 (1994); Tigges et al., 156 J. Immunol. 3901-10).

As used herein the term, “central nervous system (CNS) neurons” include the neurons of the brain, the cranial nerves and the spinal cord.

As used herein, a “chimeric protein” or “fusion protein” is a fusion of a first amino acid sequence encoding a polypeptide with a second amino acid sequence defining a domain (e.g., polypeptide portion) foreign to and not substantially homologous with the domain of the first polypeptide. A chimeric protein may present a foreign domain, which is found (albeit in a different protein) in an organism, which also expresses the first protein, or it may be an “interspecies”, “intergenic”, etc. fusion of protein structures expressed by different kinds of organisms.

As used herein, the term “contacting neurons” or “treating neurons” refers to any mode of agent delivery or “administration,” either to cells or to whole organisms, in which the agent is capable of exhibiting its pharmacological effect in neurons. “Contacting neurons” includes both in vivo and in vitro methods of bringing an agent of the invention into proximity with a neuron. Suitable modes of administration can be determined by those skilled in the art and such modes of administration may vary between agents.

As used herein, an “effective amount” of an agent or therapeutic peptide is an amount sufficient to achieve a desired therapeutic or pharmacological effect, such as an amount that is capable of inhibiting β-amyloid accumulation of Tau aggregation. An effective amount of an agent as defined herein may vary according to factors such as the disease state, age, and weight of the subject, and the ability of the agent to elicit a desired response in the subject. Dosage regimens may be adjusted to provide the optimum therapeutic response. An effective amount is also one in which any toxic or detrimental effects of the active compound are outweighed by the therapeutically beneficial effects.

As used herein, the term a “therapeutically effective amount” refers to an amount effective, at dosages and for periods of time necessary, to achieve the desired therapeutic result. A therapeutic result may be, e.g., lessening of symptoms, prolonged survival, improved mobility, and the like. A therapeutic result need not be a “cure.”

As used herein, the term “expression” refers to the process by which nucleic acid is translated into peptides or is transcribed into RNA, which, for example, can be translated into peptides, polypeptides or proteins. If the nucleic acid is derived from genomic DNA, expression may, if an appropriate eukaryotic host cell or organism is selected, include splicing of the mRNA. For heterologous nucleic acid to be expressed in a host cell, it must initially be delivered into the cell and then, once in the cell, ultimately reside in the nucleus.

As used herein, the term “genetic therapy” involves the transfer of heterologous DNA to cells of a mammal, particularly a human, with a disorder or conditions for which therapy or diagnosis is sought. The DNA is introduced into the selected target cells in a manner such that the heterologous DNA is expressed and a therapeutic product encoded thereby is produced. Alternatively, the heterologous DNA may in some manner mediate expression of DNA that encodes the therapeutic product; it may encode a product, such as a peptide or RNA that in some manner mediates, directly or indirectly, expression of a therapeutic product. Genetic therapy may also be used to deliver nucleic acid encoding a gene product to replace a defective gene or supplement a gene product produced by the mammal or the cell in which it is introduced. The introduced nucleic acid may encode a therapeutic compound that is not normally produced in the mammalian host or that is not produced in therapeutically effective amounts or at a therapeutically useful time. The heterologous DNA encoding the therapeutic product may be modified prior to introduction into the cells of the afflicted host in order to enhance or otherwise alter the product or expression thereof.

As used herein, the term “gene” or “recombinant gene” refers to a nucleic acid comprising an open reading frame encoding a polypeptide, including both exon and (optionally) intron sequences.

As used herein, the term “heterologous nucleic acid sequence” is typically DNA that encodes RNA and proteins that are not normally produced in vivo by the cell in which it is expressed or that mediates or encodes mediators that alter expression of endogenous DNA by affecting transcription, translation, or other regulatable biochemical processes. A heterologous nucleic acid sequence may also be referred to as foreign DNA. Any DNA that one of skill in the art would recognize or consider as heterologous or foreign to the cell in which it is expressed is herein encompassed by heterologous DNA. Examples of heterologous DNA include, but are not limited to, DNA that encodes traceable marker proteins, such as a protein that confers drug resistance, DNA that encodes therapeutically effective substances and DNA that encodes other types of proteins, such as antibodies. Antibodies that are encoded by heterologous DNA may be secreted or expressed on the surface of the cell in which the heterologous DNA has been introduced.

As use herein, the terms “homology” and “identity” are used synonymously throughout and refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

As used herein, the term “neurological disorder” includes a disease, disorder, or condition which directly or indirectly affects the normal functioning or anatomy of a subject's nervous system.

As used herein, the phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

As used herein, the phrases “systemic administration,” “administered systemically,” “peripheral administration” and “administered peripherally” as used herein mean the administration of a compound, drug or other material other than directly into a target tissue (e.g., the central nervous system), such that it enters the animal's system and, thus, is subject to metabolism and other like processes, for example, subcutaneous administration.

As use herein, the term “patient” or “subject” or “animal” or “host” refers to any mammal. The subject may be a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

As used herein, the term “peripheral nervous system (PNS) neurons” includes the neurons which reside or extend outside of the CNS. PNS is intended to include the neurons commonly understood as categorized in the peripheral nervous system, including sensory neurons and motor neurons.

As used herein, the terms “polynucleotide sequence” and “nucleotide sequence” are also used interchangeably herein.

As used herein, the terms “peptide” or “polypeptide” are used interchangeably herein and refer to compounds consisting of from about 2 to about 90 amino acid residues, inclusive, wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g., solid phase synthesis) or molecular biology techniques (see Sambrook et al., MOLECULAR CLONING: LAB. MANUAL (Cold Spring Harbor Press, Cold Spring Harbor, N.Y., 1989)). A “peptide” can comprise any suitable L- and/or D-amino acid, for example, common a-amino acids (e.g., alanine, glycine, valine), non-a-amino acids (e.g., P-alanine, 4-aminobutyric acid, 6aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and means for adding or removing protecting groups are known in the art. See, e.g., Green &amp; Wuts, PROTECTING GROUPS IN ORGANIC SYNTHESIS (John Wiley &amp; Sons, 1991). The functional groups of a peptide can also be derivatized (e.g., alkylated) using art-known methods.

Peptides can be synthesized and assembled into libraries comprising a few too many discrete molecular species. Such libraries can be prepared using well-known methods of combinatorial chemistry, and can be screened as described herein or using other suitable methods to determine if the library comprises peptides which can antagonize CSPG-PTPG interaction. Such peptide antagonists can then be isolated by suitable means.

As used herein, the term “peptidomimetic”, refers to a protein-like molecule designed to mimic a peptide. Peptidomimetics typically arise either from modification of an existing peptide, or by designing similar systems that mimic peptides, such as peptoids and β-peptides. Irrespective of the approach, the altered chemical structure is designed to advantageously adjust the molecular properties such as, stability or biological activity. These modifications involve changes to the peptide that do not occur naturally (such as altered backbones and the incorporation of nonnatural amino acids).

As used herein, the term “progenitor cells” are cells produced during differentiation of a stem cell that have some, but not all, of the characteristics of their terminally-differentiated progeny. Defined progenitor cells, such as “neural progenitor cells,” are committed to a lineage, but not to a specific or terminally differentiated cell type.

As used herein, the term “stem cell” means a cell that can undergo self-renewal (i.e., progeny with the same differentiation potential) and also produce progeny cells that are more restricted in differentiation potential. Within the context of the invention, a stem cell would also encompass a more differentiated cell that has dedifferentiated, for example, by nuclear transfer, by fusions with a more primitive stem cell, by introduction of specific transcription factors, or by culture under specific conditions. See, for example, Wilmut et al., Nature, 385:810-813 (1997); Ying et al., Nature, 416:545-548 (2002); Guan et al., Nature, 440:1199-1203 (2006); Takahashi et al., Cell, 126:663-676 (2006); Okita et al., Nature, 448:313-317 (2007); and Takahashi et al., Cell, 131:861-872 (2007).

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

As used herein, the term “recombinant,” as used herein, means that a protein is derived from a prokaryotic or eukaryotic expression system.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of epithelial cells. The term also covers so-called “leaky” promoters, which regulate expression of a selected nucleic acid primarily in one tissue, but cause expression in other tissues as well. The term “transfection” is used to refer to the uptake of foreign DNA by a cell. A cell has been “transfected” when exogenous DNA has been introduced inside the cell membrane. A number of transfection techniques are generally known in the art. See, e.g., Graham et al., Virology 52:456 (1973); Sambrook et al., Molecular Cloning: A Laboratory Manual (1989); Davis et al., Basic Methods in Molecular Biology (1986); Chu et al., Gene 13:197 (1981). Such techniques can be used to introduce one or more exogenous DNA moieties, such as a nucleotide integration vector and other nucleic acid molecules, into suitable host cells. The term captures chemical, electrical, and viral-mediated transfection procedures.

As used herein, the terms “transcriptional regulatory sequence” is a generic term used throughout the specification to refer to nucleic acid sequences, such as initiation signals, enhancers, and promoters, which induce or control transcription of protein coding sequences with which they are operably linked. In some examples, transcription of a recombinant gene is under the control of a promoter sequence (or other transcriptional regulatory sequence), which controls the expression of the recombinant gene in a cell-type in which expression is intended. It will also be understood that the recombinant gene can be under the control of transcriptional regulatory sequences which are the same or which are different from those sequences, which control transcription of the naturally occurring form of a protein.

As used herein, the term “vector” refers to a nucleic acid molecule capable of transporting another nucleic acid to which it has been linked. Preferred vectors are those capable of one or more of, autonomous replication and expression of nucleic acids to which they are linked. Vectors capable of directing the expression of genes to which they are operatively linked are referred to herein as “expression vectors”.

As used herein, the term “wild type” refers to the naturally-occurring polynucleotide sequence encoding a protein, or a portion thereof, or protein sequence, or portion thereof, respectively, as it normally exists in vivo. As used herein, the term “nucleic acid” refers to polynucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The agents, compounds, compositions, antibodies, etc. used in the methods described herein are considered to be purified and/or isolated prior to their use. Purified materials are typically “substantially pure”, meaning that a nucleic acid, polypeptide or fragment thereof, or other molecule has been separated from the components that naturally accompany it. Typically, the polypeptide is substantially pure when it is at least 60%, 70%, 80%, 90%, 95%, or even 99%, by weight, free from the proteins and other organic molecules with which it is associated naturally. For example, a substantially pure polypeptide may be obtained by extraction from a natural source, by expression of a recombinant nucleic acid in a cell that does not normally express that protein, or by chemical synthesis. “Isolated materials” have been removed from their natural location and environment. In the case of an isolated or purified domain or protein fragment, the domain or fragment is substantially free from amino acid sequences that flank the protein in the naturally-occurring sequence. The term “isolated DNA” means DNA has been substantially freed of the genes that flank the given DNA in the naturally occurring genome. Thus, the term “isolated DNA” encompasses, for example, cDNA, cloned genomic DNA, and synthetic DNA.

As used herein, the terms “portion”, “fragment”, “variant”, “derivative” and “analog”, when referring to a polypeptide of the present invention include any polypeptide that retains at least some biological activity referred to herein (e.g., inhibition of an interaction such as binding). Polypeptides as described herein may include portion, fragment, variant, or derivative molecules without limitation, as long as the polypeptide still serves its function. Polypeptides or portions thereof of the present invention may include proteolytic fragments, deletion fragments and in particular, or fragments that more easily reach the site of action when delivered to an animal.

Embodiments described herein relate to methods of inhibiting and/or reducing β-amyloid accumulation and/or Tau aggregation in a subject in need thereof. β-amyloid accumulation and Tau aggregation are hallmarks of Alzheimer's disease, yet their underlying molecular mechanisms remain obscure. We found that neuronal receptor PTPG mediates both β-amyloid and Tau pathogenesis in two mouse models. In the brain, PTPG binds to β-amyloid precursor protein (APP). Depletion of PTPσ reduces the affinity between APP and β-secretase, diminishing APP proteolytic products by β- and γ-cleavage without affecting other major substrates of the secretases, suggesting a specificity of β-amyloidogenic regulation. In human APP transgenic mice during aging, the progression of β-amyloidosis, Tau aggregation, neuroinflammation, synaptic loss, as well as behavioral deficits, all show unambiguous dependency on the expression of PTPσ. Additionally, the aggregates of endogenous Tau are found in a distribution pattern similar to that of early stage neurofibrillary tangles in Alzheimer brains.

It was further found that blocking PTPσ activity or function using a small peptide mimetic of the wedge shaped domain (i.e., wedge domain) of the intracellular catalytic domain of PTPσ (e.g., ISP having SEQ ID NO: 9) can suppress APP amyloidogenic processing by BACE1, to a similar degree as PTPσ depletion. Advantageously, a peptide mimetic of the wedge domain of PTPσ when delivered to a subject in need thereof can inhibit both β-amyloid and Tau pathogenesis, actively suppress β-amyloid compared to passive amyloid immunotherapies, and regulate APP processing without affecting other substrates of the secretases, thus providing a safer therapy than secretase inhibitors.

Accordingly, in some embodiments described herein, a therapeutic agent that inhibits one or more of catalytic activity, signaling, and function of a LAR family phosphatases, such as PTPσ, can be administered to a subject in need thereof to inhibit and/or reduce β-amyloid accumulation and/or Tau aggregation in the subject in need thereof and/or treat Alzheimer's disease and/or dementias related to Alzheimer's disease.

The activity, signaling, and/or function of the LAR family phosphatases, such as PTPσ, can be suppressed, inhibited, and/or blocked in several ways including: direct inhibition of the activity of the intracellular domain of the LAR family phosphatases (e.g., by using small molecules, peptidomimetics, or dominant negative polypeptides); activation of genes and/or proteins that inhibit one or more of, the activity, signaling, and/or function of the intracellular domain of the LAR family phosphatases (e.g., by increasing the expression or activity of the genes and/or proteins); inhibition of genes and/or proteins that are downstream mediators of the LAR family phosphatases (e.g., by blocking the expression and/or activity of the mediator genes and/or proteins); introduction of genes and/or proteins that negatively regulate one or more of, activity, signaling, and/or function of LAR family phosphatases (e.g., by using recombinant gene expression vectors, recombinant viral vectors or recombinant polypeptides); or gene replacement with, for instance, a hypomorphic mutant of the LAR family phosphatases (e.g., by homologous recombination, overexpression using recombinant gene expression or viral vectors, or mutagenesis).

The therapeutic agent that inhibits or reduces one or more of the activity, signaling, and/or function of the LAR family phosphatase, such as PTPσ, can include an agent that decreases and/or suppresses the activity, signaling, and/or function of the LAR family phosphatase without inhibiting binding to or activation the LAR family phosphatases by proteoglycans, such as CSPG. Such agents can be delivered intracellularly or extracellularly and once delivered to produce a neurosalutory effect.

The neurosalutary effect can include a response or result favorable to the health or function of a neuron, of a part of the nervous system, or of the nervous system generally. Examples of such effects include improvements in the ability of a neuron or portion of the nervous system to resist insult, to regenerate, to maintain desirable function, to grow or to survive. The neurosalutary effect can include producing or effecting such a response or improvement in function or resilience within a component of the nervous system. Examples of producing a neurosalutary effect would include stimulating axonal outgrowth after injury to a neuron; rendering a neuron resistant to apoptosis; rendering a neuron resistant to a toxic compound such as β-amyloid, ammonia, or other neurotoxins; reversing age-related neuronal atrophy or loss of function; reversing age-related loss of cholinergic innervation, reversing and/or reducing dieback, and/or promoting neural sprouting.

One potential mechanism for regulation, modulation, and/or inhibition of LAR family of phosphatases involves dimerization of the intracellular portion of the LAR family of phosphatases. In contrast to receptor tyrosine kinases, which are active as dimers and inactive as monomers, several protein tyrosine phosphatases (PTPs) are inactive in the dimerized state and active as monomers. These include PTPalpha, PTP1B and CD45. Each of these molecules can be crystallized in both their active monomeric form and inactive dimeric form. In addition, LAR and CD45 demonstrate homophillic binding under specific oxidative conditions, while PTPG can dimerize in response to ligand binding. This suggests that ligands to LAR family of phosphatases can direct the activation state of LAR family of phosphatase, such as LAR and PTPσ. Therefore, mimicking dimerization with intracellular-targeted therapies can directly inactivate LAR family of phosphatases without alteration of the extracellular matrix or other ligands.

In one embodiment, the therapeutic agent that inhibits or reduces one or more of the activity, signaling, and/or function of the LAR family phosphatase, such as PTPσ, can include a therapeutic peptide or small molecule that binds to and/or complexes with the intracellular domain of at least one LAR family phosphatase to inhibit the activity, signaling, and/or function of the LAR family phosphatase. Accordingly, therapeutic peptides or small molecules that binds to and/or complexes with the intracellular domain of at least one LAR family phosphatase of neural cells can be used to inhibit β-amyloid accumulation or Tau aggregation.

In some embodiments, the therapeutic agent can be a peptide mimetic of the wedged shaped domain (i.e., wedge domain) of the intracellular catalytic domain of the LAR family phosphatases. Structural and sequence analysis has revealed that all members of the LAR family contain a conserved 24 amino acid wedge-shaped helix-loop-helix motif in the first intracellular catalytic domain that can potentially mediate homo/heterophilic receptor interaction. Table 1 lists the amino acid sequences of intracellular portions of the LAR family phosphatase members that contain the wedge domain. The 24 amino acid wedge domain of these intracellular portions of LAR family phosphatases is identified by underlining. While the specific structure of the wedge domain is conserved through most LAR family wedge domains, the exact amino acids that make up the wedge domains vary between individual proteins and sub-families.

TABLE 1
LAR Wedge Domain Alignment

| | | | | |
|---|---|---|---|---|
| Mouse | 1338 | PIPITDLADNIERLKANDGKLFSQ | 1370 | SEQ ID |
| | | EYESIDPGQ | | NO: 1 |
| Rat | 1338 | PIPITDLADNIERLKANDGKLFSQ | 1370 | SEQ ID |
| | | EYESIDPGQ | | NO: 2 |
| Human | 1347 | PIPITDLADNIERLKANDGKLFSQ | 1379 | SEQ ID |
| | | EYESIDPGQ | | NO: 3 |

PTPσ Wedge Domain Alignment

| | | | | |
|---|---|---|---|---|
| Mouse | 1347 | PIPITDMAEHMERLKANDSLKLSQ | 1379 | SEQ ID |
| | | EYESIDPGQ | | NO: 4 |
| Rat | 1303 | PIPITDMAEHMERLKANDSLKLSQ | 1335 | SEQ ID |
| | | EYESIDPGQ | | NO: 5 |
| Human | 1368 | PIPIADMAEHTERLKANDSLKLSQ | 1400 | SEQ ID |
| | | EYESIDPGQ | | NO: 6 |

PTPδ Wedge Domain Alignment

| | | | | |
|---|---|---|---|---|
| Mouse | 1326 | PIPILELADHIERLKANDNLKFSQ | 1379 | SEQ ID |
| | | EYESIDPGQ | | NO: 7 |
| Human | 1335 | PIPILELADHIERLKANDNLKFSQ | 1367 | SEQ ID |
| | | EYESIDPGQ | | NO: 8 |

Wedge domains of specific LAR family members were found to engage in homophilic interaction or binding with their specific LAR family member. For example, the wedge domain of LAR was able to specifically interact with full length LAR, and not other family members such as PTPσ, in pull-down assays. In addition, in vitro binding assays showed that wedge domain peptides (wedge domain+HIV-TAT) of PTPmu and LAR specifically homophillically aggregated instead of binding promiscuously with each other. Of particular interest, the wedge domain of LAR was unable to bind to sigma, showing specificity even between similar family members.

In some embodiments, the therapeutic agent can be a peptide mimetic of the wedge shaped domain (i.e., wedge domain) of the intracellular catalytic domain of PTPσ, such as described, for example, in WO 2013/155103A1, which is herein incorporated by reference in its entirety. Peptide mimetics of the wedge domain of the PTPσ when expressed in cells (e.g., neural cells) or conjugated to an intracellular transport moiety can be used to abolish PTPσ signaling in a neural cell and inhibit β-amyloid accumulation and Tau aggregation. Binding of these therapeutic peptides to PTPσ intact wedge domain can potentially: (i) interfere with the ability for PTPσ to interact with target proteins, such as phosphatase targets; (ii) interfere with activity promoting intermolecular interactions between PTPσ and another domain contained in PTPσ, such as the catalytically inactive second phosphatase domain D2; prevent access of proteins to the active phosphatase site; (iii) out-compete normal interactors of the wedge domain; and/or (iv) sterically inhibit phosphatase activity.

In some embodiments, the peptide mimetic (i.e., therapeutic peptide) can include, consist essentially, and/or consist of about 10 to about 20 amino acids and have an amino acid sequence that is at least about 65%, at least about 70%, at least about 75%, at least about 80%, at least about 85%, at least about 90%, at least about 95%, or about 100% identical to an about 10 to about 20 consecutive amino acid portion of the amino acid sequence of the wedge domains of LAR family phosphatases, such as PTPσ.

In other embodiments, the therapeutic peptide can include, consist essentially, and/or consist of about 10 to about 20 amino acids and have an amino acid sequence that is at least about 65%, at least about 70%, at least about 75%, at least about 80%, at least about 85%, at least about 90%, at least about 95%, or about 100% identical to about 10 to about 20 consecutive amino acids of the wedge domain of PTPσ.

The wedge domain sequence of specific LAR family members is shown in Table 2.

TABLE 2
Wedge Domain Alignment

| 0 | | | | | | | | | 1 | | | | | | | | | | 2 | | | | | | |
|---|---|---|---|---|---|---|---|---|---|---|---|---|---|---|---|---|---|---|---|---|---|---|---|---|---|
| 1 | 2 | 3 | 4 | 5 | 6 | 7 | 8 | 9 | 0 | 1 | 2 | 3 | 4 | 5 | 6 | 7 | 8 | 9 | 0 | 1 | 2 | 3 | 4 | | |
| D | M | A | E | H | T | E | R | L | K | A | N | D | S | L | K | L | S | Q | E | Y | E | S | I | Human | SEQ ID NO: 9 |
| | | | | | | | | | | | | | | | | | | | | | | | | PTPσ | |
| D | L | A | D | N | I | E | R | L | K | A | N | D | G | L | K | F | S | Q | E | Y | E | S | I | LAR (Lar | SEQ ID NO: 10 |
| | | | | | | | | | | | | | | | | | | | | | | | | family) | |
| E | L | A | D | H | I | E | R | L | K | A | N | D | N | L | K | F | S | Q | E | Y | E | S | I | Delta (Lar | SEQ ID NO: 11 |
| | | | | | | | | | | | | | | | | | | | | | | | | family) | |
| K | L | E | E | E | I | N | R | R | M | A | D | D | N | K | I | F | R | E | E | F | N | A | L | ptp alpha | SEQ ID NO: 12 |

The first alpha helix of the wedge domain of PTPσ includes amino acids 1-10, the turn region includes amino acids 11-14, and the second alpha helix includes amino acids 15-24. For example, the first alpha helix of the wedge domain of human PTPσ has the amino acid sequence of DMAEHTERLK (SEQ ID NO: 13), the turn has the amino acid sequence of ANDS (SEQ ID NO: 14), and the second alpha helix has the amino acid sequence of LKLSQEYESI (SEQ ID NO: 15).

The wedge domain also shares sequence homology with the other members of the LAR family, LAR and PTPdelta. It is likely that these amino acids are necessary for the overall structure of the wedge domain. Conserved amino acids include an alanine at position 13, which marks the end of the first alpha helix and the start of the turn, making it likely to be necessary for general wedge size and structure.

Since the general secondary and tertiary structures of the wedge domain remain consistent through most receptor PTPs, several conservative substitutions can be made to a therapeutic peptide targeting the PTPσ wedge domain to obtain similar results. Examples of conservative substitutions include the substitution of one non-polar (hydrophobic) residue, such as isoleucine, valine, leucine or methionine for another, the substitution of one polar (hydrophilic) residue for another, such as between arginine and lysine, between glutamine and asparagine, between glycine and serine, the substitution of one basic residue such as lysine, arginine or histidine for another, and/or the substitution of one acidic residue, such as aspartic acid or glutamic acid for another.

These conservative substitutions can occur in the non-unique domains in either alpha helix or the turn, specifically positions 1-3 and 7-10 in the first alpha helix; 12 and 13 in the turn; and 15, 16, 18-24 in the second alpha helix. These amino acids may be necessary to the overall structure of the wedge domain, but not necessary for specificity of binding of wedge to PTPσ.

The unique amino acids to PTPσ, particularly the amino acids expressed differentially in PTPσ vs LAR, were found to be necessary for specificity of wedge domain binding. These include an EH domain in the first alpha helix position 4 and 5 followed by a threonine or a metathione (rat and mouse substitution) at position 6. In the turn, there is a unique serine at position 14 in all higher mammals. Finally, there is a unique leucine at position 17 in the second alpha helix. The potential roles of these unique amino acids will be discussed below.

The serine residue in the turn at position 14 is of particular interest due to its location in the wedge domain. This amino acid, located in the turn between alpha helixes, is slightly extended from the general secondary and tertiary structure of PTPσ, making it available for binding interactions. In addition, serine, due to its hydroxyl group and the polarity it contains, is known to facilitate several homophillic and heterophillic binding events, such as hydrogen binding between adjacent serines. Serines are also known to undergo various modifications, such as phosphorylation, making the likelihood of its necessity for specificity high. It is possible that smaller peptides that focus on the turn in the wedge domain and include the conserved serine may offer greater stability with similar function. Such peptides can be synthesized as loops, with cysteine's on either end to created di-sulfide bonds.

The unique amino acids in the first alpha helix include glutamic acid at position 4, histidine at position 5 and threonine or metathione at position 6. Although the histidine is implicated in the consensus wedge domain, it is not found in LAR, PTPdelta, PTPmu or CD45. As all three of these amino acids are either charged or polar, it is likely that either this sequence or one of its components is necessary for PTPσ wedge specificity.

Additionally, the second alpha helix contains a unique leucine at position 17. Leucines have been implicated as the critical adhesive molecules for the three dimensional structure of leucine zippers. In these molecules, which are structurally similar to wedge domains, leucines of opposing alpha helixes, located at approximately 7 intervals, interact with hydrophobic regions of the opposing alpha helix. As there is also a Leucine in the first alpha helix, located at position 9, it is believed that this unique leucine is necessary for the overall three-dimensional structural integrity of the PTPσ wedge.

Accordingly, in other embodiments, the therapeutic peptide can include, consist essentially of, or consist of about 14 to about 20 amino acids and include the amino acid sequence EHX₁ERLKANDSLKL (SEQ ID NO: 16), wherein X₁ is T or M. A therapeutic peptide including SEQ ID NO: 16 can include at least one, at least two, at least three, at least four, or at least five conservative substitutions so that the therapeutic peptide has an amino acid sequence that is at least about 65%, at least about 70%, at least about 75%, at least about 80%, at least about 85%, at least about 90%, or at least about 95% homologous to SEQ ID NO: 16.

In some embodiments, the conservative substitutions can be of amino acid residues 4E, 5R, 6L, 7K, 9N, 10D, 12L, or 13K of SEQ ID NO: 16. By way of example, amino acid residue 4E can be substituted with D or Q, amino acid residue 5R can be substituted with H, L, or K, amino acid residue 6L can be substituted with I, V, or M, amino acid residue 7K can be substituted with R or H, amino acid residue 9N can be substituted with E or D, amino acid residue 10 D can be substituted with E or N, amino acid residue 12L can be substituted with I, V, or M, and/or amino acid residue 13K can be substituted with R or H.

The therapeutic peptides described herein can be subject to other various changes, substitutions, insertions, and deletions where such changes provide for certain advantages in its use. In this regard, therapeutic peptides that bind to and/or complex with a wedge domain of the LAR family phosphatase can correspond to or be substantially homologous with, rather than be identical to, the sequence of a recited polypeptide where one or more changes are made and it retains the ability to inhibits or reduces one or more of the activity, signaling, and/or function of the LAR family phosphatase function.

The therapeutic polypeptide can be in any of a variety of forms of polypeptide derivatives, that include amides, conjugates with proteins, cyclized polypeptides, polymerized polypeptides, analogs, fragments, chemically modified polypeptides, and the like derivatives.

It will be appreciated that the conservative substitution can also include the use of a chemically derivatized residue in place of a non-derivatized residue provided that such peptide displays the requisite binding activity.

“Chemical derivative” refers to a subject polypeptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those polypeptides, which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine. Polypeptides described herein also include any polypeptide having one or more additions and/or deletions or residues relative to the sequence of a polypeptide whose sequence is shown herein, so long as the requisite activity is maintained.

One or more of peptides of the therapeutic peptides described herein can also be modified by natural processes, such as posttranslational processing, and/or by chemical modification techniques, which are known in the art. Modifications may occur in the peptide including the peptide backbone, the amino acid side-chains and the amino or carboxy termini. It will be appreciated that the same type of modification may be present in the same or varying degrees at several sites in a given peptide. Modifications comprise for example, without limitation, acetylation, acylation, addition of acetomidomethyl (Acm) group, ADP-ribosylation, amidation, covalent attachment to fiavin, covalent attachment to a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination (for reference see, Protein-structure and molecular properties, 2nd Ed., T. E. Creighton, W. H. Freeman and Company, New-York, 1993).

Peptides and/or proteins described herein may also include, for example, biologically active mutants, variants, fragments, chimeras, and analogues; fragments encompass amino acid sequences having truncations of one or more amino acids, wherein the truncation may originate from the amino terminus (N-terminus), carboxy terminus (C-terminus), or from the interior of the protein. Analogues of the invention involve an insertion or a substitution of one or more amino acids. Variants, mutants, fragments, chimeras and analogues may function as inhibitors of the LAR family phosphatases (without being restricted to the present examples).

The therapeutic polypeptides described herein may be prepared by methods known to those skilled in the art. The peptides and/or proteins may be prepared using recombinant DNA. For example, one preparation can include cultivating a host cell (bacterial or eukaryotic) under conditions, which provide for the expression of peptides and/or proteins within the cell.

The purification of the polypeptides may be done by affinity methods, ion exchange chromatography, size exclusion chromatography, hydrophobicity or other purification technique typically used for protein purification. The purification step can be performed under non-denaturating conditions. On the other hand, if a denaturating step is required, the protein may be renatured using techniques known in the art.

In some embodiments, the therapeutic peptides described herein can include additional residues that may be added at either terminus of a polypeptide for the purpose of providing a “linker” by which the polypeptides can be conveniently linked and/or affixed to other polypeptides, proteins, detectable moieties, labels, solid matrices, or carriers.

Amino acid residue linkers are usually at least one residue and can be 40 or more residues, more often 1 to 10 residues. Typical amino acid residues used for linking are glycine, tyrosine, cysteine, lysine, glutamic and aspartic acid, or the like. In addition, a subject polypeptide can differ by the sequence being modified by terminal-NH2 acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half life of the polypeptides in solutions, particularly biological fluids where proteases may be present. In this regard, polypeptide cyclization is also a useful terminal modification, and is particularly preferred also because of the stable structures formed by cyclization and in view of the biological activities observed for such cyclic peptides as described herein.

In some embodiments, the linker can be a flexible peptide linker that links the therapeutic peptide to other polypeptides, proteins, and/or molecules, such as detectable moieties, labels, solid matrices, or carriers. A flexible peptide linker can be about 20 or fewer amino acids in length. For example, a peptide linker can contain about 12 or fewer amino acid residues, e.g., 3, 4, 5, 6, 7, 8, 9, 10, 11, and 12. In some cases, a peptide linker comprises two or more of the following amino acids: glycine, serine, alanine, and threonine.

In some embodiments, a therapeutic agent comprising the therapeutic peptides described herein provide in the form of a conjugate protein or drug delivery construct includes at least a transport subdomain(s) or moiety(ies) (i.e., transport moieties) that is linked to the therapeutic peptide. The transport moieties can facilitate uptake of the therapeutic polypeptides into a mammalian (i.e., human or animal) tissue or cell (e.g., neural cell). The transport moieties can be covalently linked to the therapeutic peptides. The covalent link can include a peptide bond or a labile bond (e.g., a bond readily cleavable or subject to chemical change in the interior target cell environment). Additionally, the transport moieties can be cross-linked (e.g., chemically cross-linked, UV cross-linked) to the therapeutic polypeptide. The transport moieties can also be linked to the therapeutic peptide with linking polypeptide described herein.

The transport moieties can be repeated more than once in the therapeutic agent. The repetition of a transport moiety may affect (e.g., increase) the uptake of the peptides and/or proteins by a desired cell. The transport moiety may also be located either at the amino-terminal region of therapeutic peptide or at its carboxy-terminal region or at both regions.

In one embodiment, the transport moiety can include at least one transport peptide sequence that allows the therapeutic peptide once linked to the transport moiety to penetrate into the cell by a receptor-independent mechanism. In one example, the transport peptide is a synthetic peptide that contains a Tat-mediated protein delivery sequence and at least one of SEQ ID NOs: 9-13 and 16. These peptides can have, respectively, the amino acid sequences of SEQ ID NOs: 17-22.

Other examples of known transport moieties, subdomains and the like are described in, for example, Canadian patent document No. 2,301,157 (conjugates containing homeodomain of antennapedia) as well as in U.S. Pat. Nos. 5,652,122, 5,670,617, 5,674,980, 5,747,641, and 5,804,604, all of which are incorporated herein by reference in their entirety, (conjugates containing amino acids of Tat HIV protein; herpes simplex virus-1 DNA binding protein VP22, a Histidine tag ranging in length from 4 to 30 histidine repeats, or a variation derivative or homologue thereof capable of facilitating uptake of the active cargo moiety by a receptor independent process.

A 16 amino acid region of the third alpha-helix of antennapedia homeodomain has also been shown to enable proteins (made as fusion proteins) to cross cellular membranes (PCT international publication number WO 99/11809 and Canadian application No. 2,301,157. Similarly, HIV Tat protein was shown to be able to cross cellular membranes.

In addition, the transport moiety(ies) can include polypeptides having a basic amino acid rich region covalently linked to an active agent moiety (e.g., intracellular domain-containing fragments inhibitor peptide). As used herein, the term “basic amino acid rich region” relates to a region of a protein with a high content of the basic amino acids such as arginine, histidine, asparagine, glutamine, lysine. A “basic amino acid rich region” may have, for example 15% or more of basic amino acid. In some instance, a “basic amino acid rich region” may have less than 15% of basic amino acids and still function as a transport agent region. In other instances, a basic amino acid region will have 30% or more of basic amino acids.

The transport moiety(ies) may further include a proline rich region. As used herein, the term proline rich region refers to a region of a polypeptide with 5% or more (up to 100%) of proline in its sequence. In some instance, a proline rich region may have between 5% and 15% of prolines. Additionally, a proline rich region refers to a region, of a polypeptide containing more prolines than what is generally observed in naturally occurring proteins (e.g., proteins encoded by the human genome). Proline rich regions of this application can function as a transport agent region.

In one embodiment, the therapeutic peptide described herein can be non-covalently linked to a transduction agent. An example of a non-covalently linked polypeptide transduction agent is the Chariot protein delivery system (See U.S. Pat. No. 6,841,535; J Biol Chem 274(35):24941-24946; and Nature Biotec. 19:1173-1176, all herein incorporated by reference in their entirety).

In other embodiments, the therapeutic peptides can be expressed in cells being treated using gene therapy to inhibit LAR family signaling. The gene therapy can use a vector including a nucleotide encoding the therapeutic peptides. A “vector” (sometimes referred to as gene delivery or gene transfer “vehicle”) refers to a macromolecule or complex of molecules comprising a polynucleotide to be delivered to the cell. The polynucleotide to be delivered may comprise a coding sequence of interest in gene therapy. Vectors include, for example, viral vectors (such as adenoviruses (Ad), adeno-associated viruses (AAV), and retroviruses), liposomes and other lipid-containing complexes, and other macromolecular complexes capable of mediating delivery of a polynucleotide to a target cell.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities.

Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., Lupton, S., WO 92/08796, published May 29, 1992; and Lupton, S., WO 94/28143, published Dec. 8, 1994). Such marker genes can provide an added measure of control that can be advantageous in gene therapy contexts. A large variety of such vectors are known in the art and are generally available.

Vectors for use herein include viral vectors, lipid based vectors and other non-viral vectors that are capable of delivering a nucleotide encoding the therapeutic peptides described herein to the target cells. The vector can be a targeted vector, especially a targeted vector that preferentially binds to neurons and. Viral vectors for use in the application can include those that exhibit low toxicity to a target cell and induce production of therapeutically useful quantities of the therapeutic peptide in a cell specific manner.

Examples of viral vectors are those derived from adenovirus (Ad) or adeno-associated virus (AAV). Both human and non-human viral vectors can be used and the recombinant viral vector can be replication-defective in humans. Where the vector is an adenovirus, the vector can comprise a polynucleotide having a promoter operably linked to a gene encoding the therapeutic peptides and is replication-defective in humans.

Other viral vectors that can be used herein include herpes simplex virus (HSV)-based vectors. HSV vectors deleted of one or more immediate early genes (IE) are advantageous because they are generally non-cytotoxic, persist in a state similar to latency in the target cell, and afford efficient target cell transduction. Recombinant HSV vectors can incorporate approximately 30 kb of heterologous nucleic acid.

Retroviruses, such as C-type retroviruses and lentiviruses, might also be used in the application. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and a nucleic acid encoding the therapeutic peptide. In methods of delivery to neural cells, it may also encode a ligand to a tissue specific receptor.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells.

Lentiviral vectors for use in the application may be derived from human and non-human (including SIV) lentiviruses. Examples of lentiviral vectors include nucleic acid sequences required for vector propagation as well as a tissue-specific promoter operably linked to a therapeutic peptide encoding nucleic acid. These former may include the viral LTRs, a primer binding site, a polypurine tract, att sites, and an encapsidation site.

In some aspects, a lentiviral vector can be employed. Lentiviruses have proven capable of transducing different types of CNS neurons (Azzouz et al., (2002) J Neurosci. 22: 10302-12) and may be used in some embodiments because of their large cloning capacity.

A lentiviral vector may be packaged into any lentiviral capsid. The substitution of one particle protein with another from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN) might also be used in the application. Use of alphaviruses is described in Lundstrom, K., Intervirology 43:247-257, 2000 and Perri et al., Journal of Virology 74:9802-9807, 2000.

Recombinant, replication-defective alphavirus vectors are advantageous because they are capable of high-level heterologous (therapeutic) gene expression, and can infect a wide target cell range. Alphavirus replicons may be targeted to specific cell types by displaying on their virion surface a functional heterologous ligand or binding domain that would allow selective binding to target cells expressing a cognate binding partner. Alphavirus replicons may establish latency, and therefore long-term heterologous nucleic acid expression in a target cell. The replicons may also exhibit transient heterologous nucleic acid expression in the target cell.

In many of the viral vectors compatible with methods of the application, more than one promoter can be included in the vector to allow more than one heterologous gene to be expressed by the vector. Further, the vector can comprise a sequence, which encodes a signal peptide or other moiety, which facilitates expression of the therapeutic peptide from the target cell.

To combine advantageous properties of two viral vector systems, hybrid viral vectors may be used to deliver a nucleic acid encoding a therapeutic peptide to a target neuron, cell, or tissue. Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186, 2000. Retroviral genomes contained within an adenovirus may integrate within the target cell genome and effect stable gene expression.

Other nucleotide sequence elements, which facilitate expression of the therapeutic peptide and cloning of the vector are further contemplated. For example, the presence of enhancers upstream of the promoter or terminators downstream of the coding region, for example, can facilitate expression.

In accordance with another embodiment, a tissue-specific promoter can be fused to nucleotides encoding the therapeutic peptides described herein. By fusing such tissue specific promoter within the adenoviral construct, transgene expression is limited to a particular tissue. The efficacy of gene expression and degree of specificity provided by tissue specific promoters can be determined, using the recombinant adenoviral system of the present application. Neuron specific promoters, such as the platelet-derived growth factor β-chain (PDGF-0) promoter and vectors, are well known in the art.

In addition to viral vector-based methods, non-viral methods may also be used to introduce a nucleic acid encoding a therapeutic peptide into a target cell. A review of non-viral methods of gene delivery is provided in Nishikawa and Huang, Human Gene Ther. 12:861-870, 2001. An example of a non-viral gene delivery method according to the application employs plasmid DNA to introduce a nucleic acid encoding a therapeutic peptide into a cell. Plasmid-based gene delivery methods are generally known in the art.

Synthetic gene transfer molecules can be designed to form multimolecular aggregates with plasmid DNA. These aggregates can be designed to bind to a target cell. Cationic amphiphiles, including lipopolyamines and cationic lipids, may be used to provide receptor-independent nucleic acid transfer into target cells.

In addition, preformed cationic liposomes or cationic lipids may be mixed with plasmid DNA to generate cell-transfecting complexes. Methods involving cationic lipid formulations are reviewed in Felgner et al., Ann. N.Y. Acad. Sci. 772:126-139, 1995 and Lasic and Templeton, Adv. Drug Delivery Rev. 20:221-266, 1996. For gene delivery, DNA may also be coupled to an amphipathic cationic peptide (Fominaya et al., J. Gene Med. 2:455-464, 2000).

Methods that involve both viral and non-viral based components may be used according to the application. For example, an Epstein Barr virus (EBV)-based plasmid for therapeutic gene delivery is described in Cui et al., Gene Therapy 8:1508-1513, 2001. Additionally, a method involving a DNA/ligand/polycationic adjunct coupled to an adenovirus is described in Curiel, D. T., Nat. Immun. 13:141-164, 1994.

Additionally, the nucleic acid encoding the therapeutic peptides can be introduced into the target cell by transfecting the target cells using electroporation techniques. Electroporation techniques are well known and can be used to facilitate transfection of cells using plasmid DNA.

Vectors that encode the expression of the therapeutic peptides can be delivered in vivo to the target cell in the form of an injectable preparation containing pharmaceutically acceptable carrier, such as saline, as necessary. Other pharmaceutical carriers, formulations and dosages can also be used in accordance with the present application.

Where the target cell includes a neuron being treated, such as quiescent or dormant neurons, the vector can be delivered by direct injection at an amount sufficient for the therapeutic peptide to be expressed to a degree, which allows for highly effective therapy. By injecting the vector directly into or about the periphery of the neuron, it is possible to target the vector transfection rather effectively, and to minimize loss of the recombinant vectors. This type of injection enables local transfection of a desired number of cells, especially at a site of CNS injury, thereby maximizing therapeutic efficacy of gene transfer, and minimizing the possibility of an inflammatory response to viral proteins. Other methods of administering the vector to the target cells can be used and will depend on the specific vector employed.

The therapeutic peptide can be expressed for any suitable length of time within the target cell, including transient expression and stable, long-term expression. In one aspect of the application, the nucleic acid encoding the therapeutic peptide will be expressed in therapeutic amounts for a defined length of time effective to induce activity and growth of the transfected cells. In another aspect of the application, the nucleic acid encoding the therapeutic peptide will be expressed in therapeutic amounts for a defined length of time effective to in inhibit and/or reduce β-amyloid accumulation and/or Tau aggregation in a subject in need thereof.

A therapeutic amount is an amount, which is capable of producing a medically desirable result in a treated animal or human. As is well known in the medical arts, dosage for any one animal or human depends on many factors, including the subject's size, body surface area, age, the particular composition to be administered, sex, time and route of administration, general health, and other drugs being administered concurrently. Specific dosages of proteins and nucleic acids can be determined readily determined by one skilled in the art using the experimental methods described below.

The therapeutic agents described herein may further be modified (e.g., chemically modified). Such modification may be designed to facilitate manipulation or purification of the molecule, to increase solubility of the molecule, to facilitate administration, targeting to the desired location, to increase or decrease half life. A number of such modifications are known in the art and can be applied by the skilled practitioner.

In some embodiments, the therapeutic agents and pharmaceutical compositions comprising the therapeutic agents described herein may be delivered to neurons of the CNS and/or the PNS. Such neurons may be injured or diseased. Such neurons may alternatively be healthy, uninjured neurons. Such neurons may be located at the site of injury, or at a site incident to the injury. The neurons to be targeted for therapeutic administration, delivery/contact of the agents and compositions described herein will be neurons from which neuronal outgrowth is thought to prove beneficial to the subject. Such determination is within the ability of the skilled practitioner through no more than routine experimentation.

The therapeutic agents and therapeutic pharmaceutical compositions described herein may also be delivered to non-neuronal cells of the CNS and/or the PNS, such as to non-neuronal cells that provide support to neural cells. Such cells include, without limitation, glial cells (e.g., astrocytes, oligodendrocytes, ependymal cells, radial glia in the CNS; and Schwann cells, satellite glial cells, enteric glial cells n the PNS).

In one embodiment, the administration is specific for one or more specific locations within the subject's nervous system. The preferred mode of administration can vary depending upon the particular agent chosen and the particular target.

When the therapeutic agents are delivered to a subject, they can be administered by any suitable route, including, for example, orally (e.g., in capsules, suspensions or tablets), systemically, or by parenteral administration. Parenteral administration can include, for example, intramuscular, intravenous, intraarticular, intraarterial, intrathecal, subcutaneous, or intraperitoneal administration. The agent can also be administered orally, transdermally, topically, by inhalation (e.g., intrabronchial, intranasal, oral inhalation or intranasal drops) or rectally. Administration can be local or systemic as indicated.

Both local and systemic administration are contemplated herein. Desirable features of local administration include achieving effective local concentrations of the therapeutic agent as well as avoiding adverse side effects from systemic administration of the therapeutic agent. In one embodiment, the therapeutic agent can be administered by introduction into the cerebrospinal fluid of the subject. In certain aspects, the therapeutic agent can be introduced into a cerebral ventricle, the lumbar area, or the cistema magna. In another aspect, the therapeutic agent can be introduced locally, such as into the site of nerve or cord injury, into a site of pain or neural degeneration, or intraocularly to contact neuroretinal cells.

The pharmaceutically acceptable formulations can be suspended in aqueous vehicles and introduced through conventional hypodermic needles or using infusion pumps.

In another embodiment, the therapeutic agent can be administered into a subject intrathecally. As used herein, the term “intrathecal administration” is intended to include delivering a therapeutic agent directly into the cerebrospinal fluid of a subject, by techniques including lateral cerebroventricular injection through a burrhole or cisternal or lumbar puncture or the like (described in Lazorthes et al., 1991, and Ommaya, 1984, the contents of which are incorporated herein by reference). The term “lumbar region” is intended to include the area between the third and fourth lumbar (lower back) vertebrae. The term “cistema magna” is intended to include the area where the skull ends and the spinal cord begins at the back of the head. The ten-n “cerebral ventricle” is intended to include the cavities in the brain that are continuous with the central canal of the spinal cord. Administration of therapeutic agent to any of the above mentioned sites can be achieved by direct injection of the therapeutic agent or by the use of infusion pumps. Implantable or external pumps and catheter may be used.

For injection, therapeutic agent can be formulated in liquid solutions, typically in physiologically compatible buffers such as Hank's solution or Ringer's solution. In addition, the therapeutic agent may be formulated in solid form and re-dissolved or suspended immediately prior to use. Lyophilized forms are also included. The injection can be, for example, in the form of a bolus injection or continuous infusion (such as using infusion pumps) of the therapeutic agent.

In one embodiment, the therapeutic agent can be administered by lateral cerebroventricular injection into the brain of a subject. The injection can be made, for example, through a burr hole made in the subject's skull. In another embodiment, the therapeutic agent can be administered through a surgically inserted shunt into the cerebral ventricle of a subject. For example, the injection can be made into the lateral ventricles, which are larger, even though injection into the third and fourth smaller ventricles can also be made. In yet another embodiment, the therapeutic agent can be administered by injection into the cistema magna, or lumbar area of a subject.

An additional means of administration to intracranial tissue involves application to the olfactory epithelium, with subsequent transmission to the olfactory bulb and transport to more proximal portions of the brain. Such administration can be by nebulized or aerosolized preparations.

In another embodiment, the therapeutic agent can be administered to a subject at the site of injury or systemically to the subject.

In some embodiments, the therapeutic agent can administered to a subject for an extended period of time. Sustained contact with the active compound can be achieved, for example, by repeated administration of the active compound(s) over a period of time, such as one week, several weeks, one month or longer. The pharmaceutically acceptable formulation used to administer the therapeutic agent(s) can also be formulated to provide sustained delivery of the active compound to a subject. For example, the formulation may deliver the active compound for at least one, two, three, or four weeks, inclusive, following initial administration to the subject. For example, a subject to be treated in accordance with the present invention is treated with the active compound for at least 30 days (either by repeated administration or by use of a sustained delivery system, or both).

Sustained delivery of the therapeutic agent can be demonstrated by, for example, the continued therapeutic effect of the therapeutic agent over time. Alternatively, sustained delivery of the therapeutic agent may be demonstrated by detecting the presence of the therapeutic agents in vivo over time.

Approaches for sustained delivery include use of a polymeric capsule, a minipump to deliver the formulation, a biodegradable implant, or implanted transgenic autologous cells (see U.S. Pat. No. 6,214,622). Implantable infusion pump systems (e.g., INFUSAID pumps (Towanda, Pa.)); see Zierski et al., 1988; Kanoff, 1994) and osmotic pumps (sold by Alza Corporation) are available commercially and otherwise known in the art. Another mode of administration is via an implantable, externally programmable infusion pump. Infusion pump systems and reservoir systems are also described in, e.g., U.S. Pat. Nos. 5,368,562 and 4,731,058.

Vectors encoding the therapeutic peptides can often be administered less frequently than other types of therapeutics. For example, an effective amount of such a vector can range from about 0.01 mg/kg to about 5 or 10 mg/kg, inclusive; administered daily, weekly, biweekly, monthly or less frequently.

The ability to deliver or express the therapeutic peptides allows for cell activity modulation in a number of different cell types. The therapeutic peptides can be expressed, for example, in neural cells or brain areas affected by degenerative diseases, such as Alzheimer's disease.

In some embodiments, the therapeutic agents can be used to treat diseases, disorders, or condition associated with β-amyloid accumulation and/or Tau aggregation in a subject in need thereof.

In some embodiments, the disease, disorder, and/or condition includes at least one of a disease, disorder, and/or condition of the nervous system.

In other embodiments, the disease, disorder, and/or condition of the nervous system includes at least one of a neurological disorder, neuropsychiatric disorder, neural injury, neural toxicity disorder, a neuropathic pain, and neural degenerative disorders.

For example, the neurological disorder can include at least one of Alzheimer's disease or dementias related to Alzheimer's disease.

In other embodiments, the therapeutic agent described herein can be administered in combination with an anti-Alzheimer's agent. The term “anti-Alzheimer's agent” or “anti-Alzheimer agent”, as employed herein refers to any compound that can be employed for the treatment of Alzheimer's disease and other dementias; such as, but not limited to, N-methyl-D-aspartate receptor (NMDA) receptor antagonists, acetyl cholinesterase inhibitor, acetylcholine synthesis modulators, acetylcholine storage modulators, acetylcholine release modulators, Aβ inhibitors, Aβ plaque removal agents, inhibitors of Aβ plaque formation, inhibitors of amyloid precursor protein processing enzymes, β-amyloid converting enzyme inhibitors, β-secretase inhibitors, γ-secretase modulators, nerve growth factor agonists, hormone receptor blockade agents, neurotransmission modulators, and combinations thereof. In one embodiment, the anti-Alzheimer's agent is an NMDA receptor antagonist. In one embodiment, the NMDA receptor antagonist includes, but not limited to, memantine, amantadine, neramexane (1, 3, 3, 5, 5-pentamethylcyclohexan-1-amine), ketamine, rimantidine, eliprodil, ifenprodil, dizocilpine, remacemide, riluzole, aptiganel, phencyclidine, flupirtine, celfotel, felbamate, spermine, spermidine, levemopamil, and/or combinations thereof. In another embodiment, NMDA receptor antagonist employed in the present invention is an Anti-Alzheimer agent. In one embodiment, the anti-Alzheimer's agent is an inhibitor of cholinesterase. In one embodiment, the acetylcholinesterase inhibitor includes, but is not limited to, donepezil, tacrine, rivastigmine, galantamine, physostigmine, neostigmine, Huperzine A, icopezil (CP-118954, 5,7-dihydro-3-[2-[1-(phenylmethyl)-4-piperidinyl]ethyl]-6H-pyrrolo-[4,5-f-]-1,2-benzisoxazol-6-one maleate), ER-127528 (4-[(5,6-dimethoxy-2-fluoro-1-indanon)-2-yl]methyl-1-(3-fluorobenzyl) piperidine hydrochloride), zanapezil (TAK-147; 3-[1-(phenylmethyl)piperidin-4-yl]-1-(2,3,4,5-tetrahydro-1H-1-benzazepin-8-yl)-1-propane fumarate), metrifonate (T-588; -) (—R-α-[[2-(dimethylamino)ethoxy]methyl]benzo[b]thiophene-5-methano-1 hydrochloride), FK-960 (N-(4-acetyl-1-piperazinyl)-p-fluorobenzamide-hydrate), TCH-346 (N-methyl-N-2-pyropinyldibenz[b,f]oxepine-10-methanamine), SDZ-220-581 ((S)-.alpha.-amino-5-(phosphonomethyl)-[1,1′-biphenyl]-3-propionic acid), and combinations thereof.

In another embodiment, the anti-Alzheimer's agent is an A13 inhibitor, A13 plaque removal agents, inhibitors of Aβ plaque formation, inhibitors of amyloid precursor protein processing enzymes, β-amyloid converting enzyme inhibitors, β-secretase inhibitors, γ-secretase modulators.

In another embodiment, the Aβ inhibitor includes, but is not limited to, tarenflurbil, tramiprosate, clioquinol, PBT-2 and other 8-hydroxyquinilone derivatives, Aβ plaque removal agents, inhibitors of Aβ plaque formation, inhibitors of amyloid precursor protein processing enzymes, β-amyloid converting enzyme inhibitors, β-secretase inhibitors, α-secretase modulators (LY450139; N—[N-(3,5-difluorophenacetyl)-L-alanyl)-S-phenylglycine t-butyl ester), and combinations thereof.

In another embodiment, the anti-Alzheimer's agent is a nerve growth factor agonist. The nerve growth factor agonist is, but not limited to, xaliproden or brain derived neurotrophic factor or nerve growth factor.

In another embodiment, the anti-Alzheimer's agent is a hormone receptor blockade agent. The hormone receptor blockade agent is, but not limited to, leuproelide or a derivative thereof.

In another embodiment, the anti-Alzheimer's agent is a neurotransmission modulator. The neurotransmission modulator is, but not limited to, ispronicline.

The invention is further illustrated by the following example, which is not intended to limit the scope of the claims.

### Example 1

The Leukocyte-common Antigen-Related (LAR) family of phosphatases consists of three members: LAR itself, receptor protein tyrosine phosphatase Sigma (RPTPσ) and receptor protein tyrosine phosphatase delta (RPTPδ). Structural and sequence analysis has revealed that all members of the LAR family contain a wedge-shaped helix-loop-helix motif in the first intracellular catalytic domain that mediates homo/heterophilic receptor interaction. Using peptide mimetics of this wedge domain tagged to a cytosolic localizing TAT sequence, LAR activity was successfully abolished in neurotrophin signaling paradigms. We utilized NIH BLAST to identify the orthologous sequence in RPTPσ and RPTPB and designed a wedge domain peptide for each target. The peptides were coined Intraceullar LAR blocking peptide (ILP), intracellular Sigma blocking peptide (ISP) and intracellular delta blocking peptide (IDP). Interestingly, this domain is highly conserved among higher vertebrates, indicating a functionally important region. The peptides was tagged conjugated to HIV-TAT to create function blocking peptides:

(SEQ ID NO: 17)
NH₂GRKKRRQRRRCDMAEHTMERLKANDSLKLSQEYESI-NH₂PTPσ
human (ISP).
(SEQ ID NO: 18)
NH₂GRKKRRQRRRCDLADNIERLKANDGLKFSQEYESI-NH_{S}LAR(ILP).
(SEQ ID NO: 19)
NH₂GRKKRRQRRRCELADHIERLKANDNLKFSQEYESI-NH₂PTPdelta
(IDP).

### Example 2

This example shows that inhibition of neuronal receptor PTPσ (protein tyrosine phosphatase sigma) curbs β-amyloid (Aβ) pathogenesis and aggregation of Tau. Genetic depletion of PTPσ lowers β-secretase affinity to APP and suppresses Aβ accumulation in a specific manner that does not generically inhibit β- and γ-secretase activities. Genetic depletion of PTPσ also inhibits the aggregation of Tau.

In the two mouse models described herein, a spectrum of AD neuropathologies and behavioral deficits all demonstrate a clear dependency on PTPσ, indicating that this neuronal receptor is a pivotal upstream player in AD pathogenesis.

The data suggests that targeting PTPσ is a potential therapeutic approach that canovercome such dominant genetic driving forces to curtail AD progression. The advantage of this targeting strategy is that it suppresses Aβ accumulation without broadly affecting other major substrates of the β- and γ-secretases, thus predicting a more promising translational potential as compared to those in clinical trials that generically inhibit the secretases.

### Materials and Methods

### Mouse Lines:

Mice were maintained under standard conditions approved by the Institutional Animal Care and Use Committee. Wild type and PTPσ-deficient mice of Balb/c background were provided by Dr. Michel L. Tremblay. Homozygous TgAPP-SwDI mice, C57BL/6-Tg(Thy1-APPSwDutIowa)BWevn/Mmjax, stock number 007027, were from the Jackson Laboratory. These mice express human APP transgene harboring Swedish, Dutch, and Iowa mutations, and were bred with Balb/c mice heterozygous for the PTPG gene to generate bigenic mice heterozygous for both TgAPP-SwDI and PTPG genes, which are hybrids of 50% C57BL/6J and 50% Balb/c genetic background. These mice were further bred with Balb/c mice heterozygous for the PTPσ gene. The offspring from this mating are used in experiments, which include littermates of the following genotypes: TgAPP-SwDI(+/−)PTPσ(+/+), mice heterozygous for TgAPP-SwDI transgene with wild type PTPσ; TgAPP-SwDI(+/−)PTPσ(−/−), mice heterozygous for TgAPP-SwDI transgene with genetic depletion of PTPσ; TgAPP-SwDI(−/−)PTPσ(+/+), mice free of TgAPP-SwDI transgene with wild type PTPσ. Both TgAPP-SwDI(−/−)PTPσ(+/+) and Balb/c PTPσ(+/+) are wild type mice but with different genetic background. Heterozygous TgAPP-SwInd (J20) mice, 6.Cg-Tg(PDGFB-APPSwInd)20Lms/2Mmjax, were provided by Dr. Lennart Mucke. These mice express human APP transgene harboring Swedish and Indiana mutations, and were bred with the same strategy as described above to obtain mice with genotypes of TgAPP-SwInd (+/−)PTPσ(+/+) and TgAPP-SwInd (+/−)PTPσ(−/−).

### Immunohistochemistry

Adult rat and mice were perfused intracardially with fresh made 4% paraformaldehyde in cold phosphate-buffered saline (PBS). The brains were collected and post-fixed for 2 days at 4° C. Paraffin embedded sections of 10 μM thickness were collected for immunostaining. The sections were deparaffinized and sequentially rehydrated. Antigen retrieval was performed at 100° C. in Tris-EDTA buffer (pH 9.0) for 50 min. Sections were subsequently washed with distilled water and PBS, incubated at room temperature for 1 hour in blocking buffer (PBS, with 5% normal donkey serum, 5% normal goat serum, and 0.2% Triton X-100). Primary antibody incubation was performed in a humidified chamber at 4° C. overnight. After 3 washes in PBS with 0.2% Triton X-100, the sections were then incubated with a mixture of secondary and tertiary antibodies at room temperature for 2 hours. All antibodies were diluted in blocking buffer with concentrations recommended by the manufacturers. Mouse primary antibodies were detected by goat anti-mouse Alexa488 together with donkey anti-goat Alexa488 antibodies; rabbit primary antibodies were detected by chicken anti-rabbit CF568 and donkey anti-chicken Cy3 antibodies; chicken antibody was detected with donkey anti-chicken Cy3 antibody. Sections stained with only secondary and tertiary antibodies (without primary antibodies) were used as negative controls. At last, DAPI (Invitrogen, 300 nM) was applied on sections for nuclear staining. Sections were washed 5 times before mounted in Fluoromount (SouthernBiotech).

Wide field and confocal images were captured using Zeiss Axio Imager M2 and LSM780, respectively. Images are quantified using the Zen 2 Pro software and ImageJ.

### Protein Extraction, Immunoprecipitation, and Western Blot Analysis

For the co-immunoprecipitation of APP and PTPσ, RIPA buffer was used (50 mM Tris-HCl, pH 8.0, 1 mM EDTA, 150 mM NaCl, 1% NP40, 0.1% SDS, 0.5% sodium deoxycholate). For the co-immunoprecipitation of APP and BACE1, NP40 buffer was used (50 mM Tris-HCl, pH 8.0, 1 mM EDTA, 150 mM NaCl, 1% NP40) without or with SDS at concentration of 0.1%, 0.3%, and 0.4%. For total protein extraction and immunopurification of CTFβ, SDS concentration in RIPA buffer was adjusted to 1% to ensure protein extraction from the lipid rafts. Mouse or rat forebrains were homogenized thoroughly on ice in homogenization buffers (as mention above) containing protease and phosphatase inhibitors (Thermo Scientific). For each half of forebrain, buffer volume of at least 5 ml for mouse and 8 ml for rat was used to ensure sufficient detergent/tissue ratio. The homogenates were incubated at 4° C. for 1 hour with gentle mixing, sonicated on ice for 2 minutes in a sonic dismembrator (Fisher Scientific Model 120, with pulses of 50% output, 1 second on and 1 second off), followed with another hour of gentle mixing at 4° C. All samples were used fresh without freezing and thawing.

For co-immunoprecipitation and immunopurification, the homogenates were then centrifuged at 85,000×g for 1 hour at 4° C. and the supernatants were collected. Protein concentration was measured using BCA Protein Assay Kit (Thermo Scientific). 0.5 mg total proteins of brain homogenates were incubated with 5 μg of designated antibody and 30 μl of Protein-A sepharose beads (50% slurry, Roche), in a total volume of 1 ml adjusted with RIPA buffer. Samples were gently mixed at 4° C. overnight. Subsequently, the beads were washed 5 times with cold immunoprecipitation buffer. Samples were then incubated in Laemmli buffer with 100 mM of DTT at 75° C. for 20 minutes and subjected to western blot analysis.

For analysis of protein expression level, the homogenates were centrifuged at 23,000×g for 30 min at 4° C. and the supernatants were collected. Protein concentration was measured using BCA Protein Assay Kit (Thermo Scientific). 30 μg of total proteins were subjected to western blot analysis.

Electrophoresis of protein samples was conducted using 4-12% Bis-Tris Bolt Plus Gels, with either MOPS or MES buffer and Novex Sharp Pre-stained Protein Standard (all from Invitrogen). Proteins were transferred to nitrocellulose membrane (0.2 m pore size, Bio-Rad) and blotted with selected antibodies (see table above) at concentrations suggested by the manufacturers. Primary antibodies were diluted in SuperBlock TBS Blocking Buffer (Thermo Scientific) and incubated with the nitrocellulose membranes at 4° C. overnight; secondary antibodies were diluted in PBS with 5% nonfat milk and 0.2% Tween20 and incubated at room temperature for 2 hours. Membranes were washes 4 times in PBS with 0.2% Tween20 between primary and secondary antibodies and before chemiluminescent detection with SuperSignal West Pico Chemiluminescent Substrate (Thermo Scientific).

Western blot band intensity was quantified by densitometry.

### Aβ ELISA Assays

Mouse forebrains were thoroughly homogenized in tissue homogenization buffer (2 mM Tris pH 7.4, 250 mM sucrose, 0.5 mM EDTA, 0.5 mM EGTA) containing protease inhibitor cocktail (Roche), followed by centrifugation at 135,000×g (33,500 RPM with SW50.1 rotor) for 1 hour at 4° C. Proteins in the pellets were extracted with formic acid (FA) and centrifuged at 109,000×g (30,100 RPM with SW50.1 rotor) for 1 hour at 4° C. The supernatants were collected and diluted 1:20 in neutralization buffer (1 M Tris base, 0.5 M Na₂HPO₄, 0.05% NaN₃) and subsequently 1:3 in ELISA buffer (PBS with 0.05% Tween-20, 1% BSA, and 1 mM AEBSF). Diluted samples were loaded onto ELISA plates pre-coated with 6E10 antibody (Biolegend) to capture Aβ peptides. Serial dilutions of synthesized human Aβ 1-40 or 1-42 (American Peptide) were loaded to determine a standard curve. Aβ was detected using an HRP labeled antibody for either Aβ 1-40 or 1-42 (see table above). ELISA was developed using TMB substrate (Thermo Scientific) and reaction was stopped with 1N HCl. Plates were read at 450 nm and concentrations of A1 in samples were determined using the standard curve.

### Behavior Assays

### The Y-Maze Assay

Mice were placed in the center of the Y-maze and allowed to move freely through each arm. Their exploratory activities were recorded for 5 minutes. An arm entry is defined as when all four limbs are within the arm. For each mouse, the number of triads is counted as “spontaneous alternation”, which was then divided by the number of total arm entries, yielding a percentage score. The novel object test: On day 1, mice were exposed to empty cages (45 cm×24 cm×22 cm) with blackened walls to allow exploration and habituation to the arena. During day 2 to day 4, mice were returned to the same cage with two identical objects placed at an equal distance. On each day mice were returned to the cage at approximately the same time during the day and allowed to explore for 10 minutes. Cages and objects were cleaned with 70% ethanol between each animal. Subsequently, 2 hours after the familiarization session on day 4, mice were put back to the same cage where one of the familiar objects (randomly chosen) was replaced with a novel object, and allowed to explore for 5 minutes. Mice were scored using Observer software (Noldus) on their time duration and visiting frequency exploring either object. Object exploration was defined as facing the object and actively sniffing or touching the object, whereas any climbing behavior was not scored. The discrimination indexes reflecting interest in the novel object is denoted as either the ratio of novel object exploration to total object exploration (NO/NO+FO) or the ratio of novel object exploration to familiar object exploration (NO/FO). All tests and data analyses were conducted in a double-blinded manner.

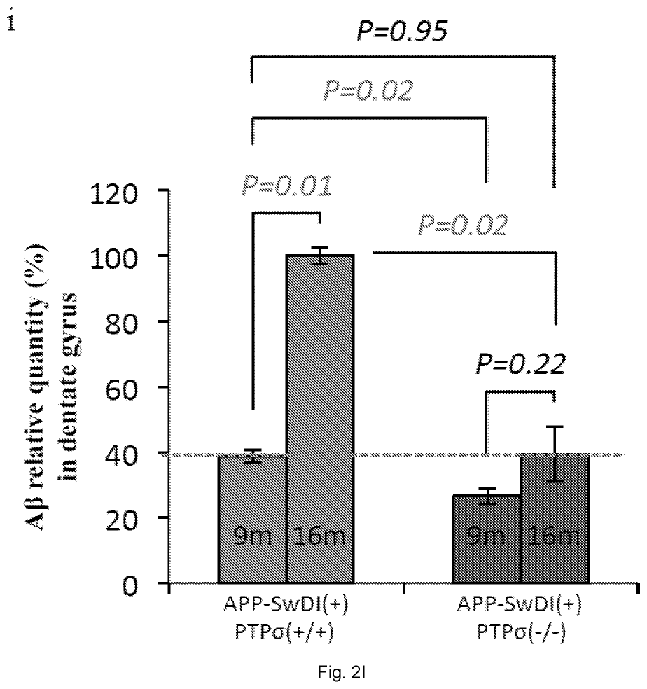

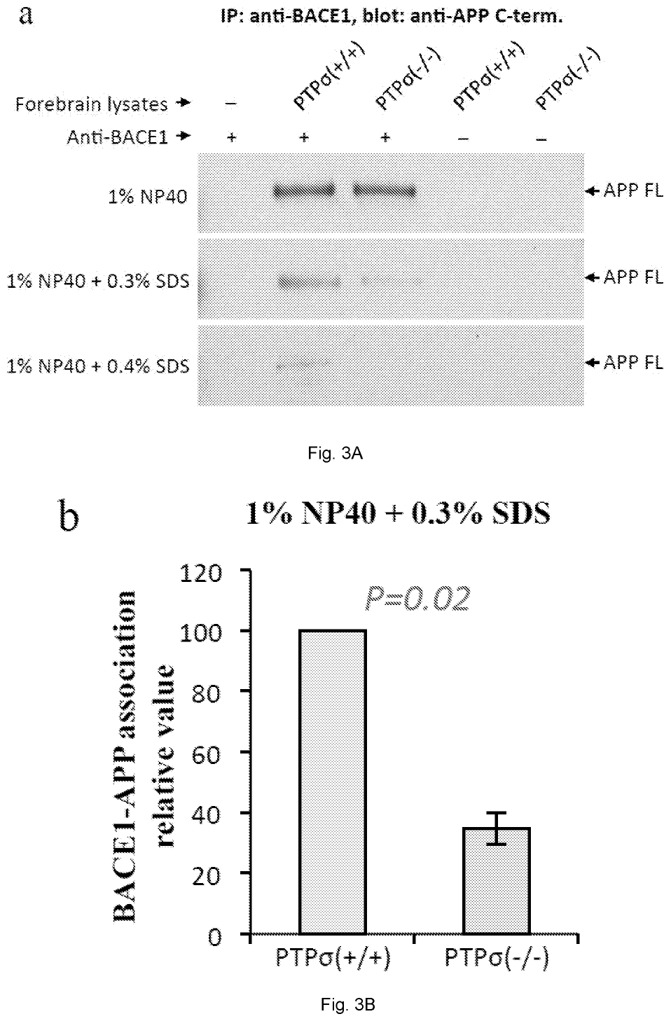

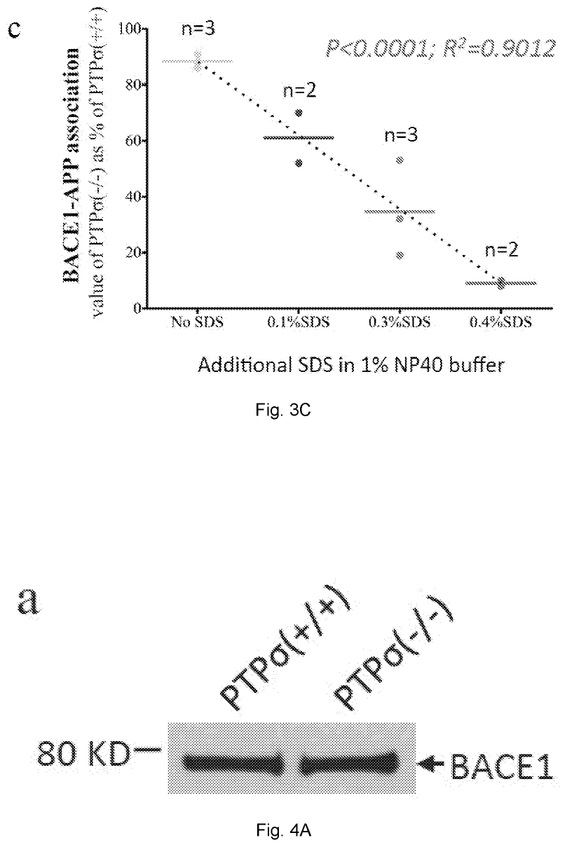

2-tailed Student's t test was used for two-group comparison. Relationship between two variables (SDS concentration and APP-BACE1 association, as in FIG. 3) was analyzed using linear regression. All error bars show standard error of the means (SEM).

### PTPσ is an APP Binding Partner in the Brain

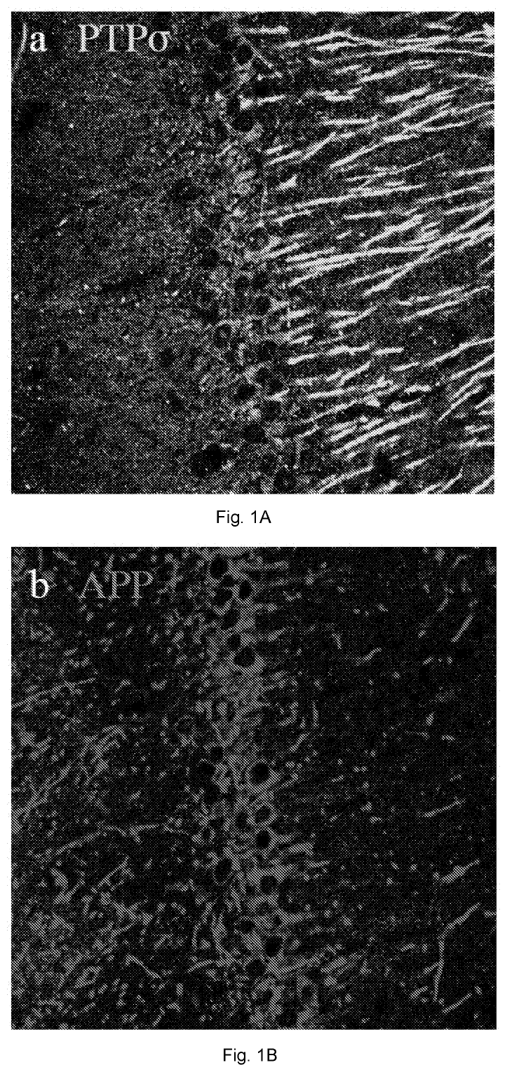

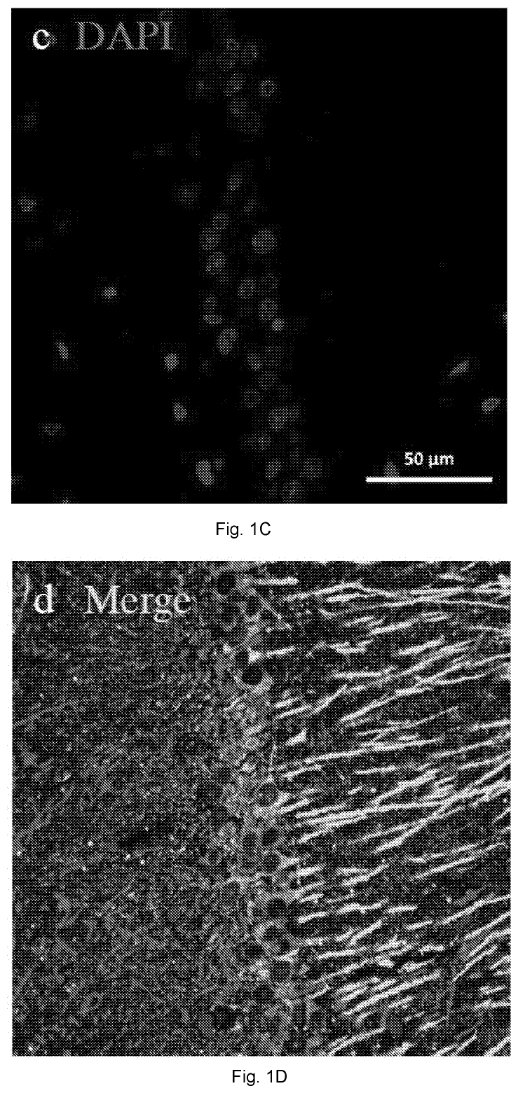

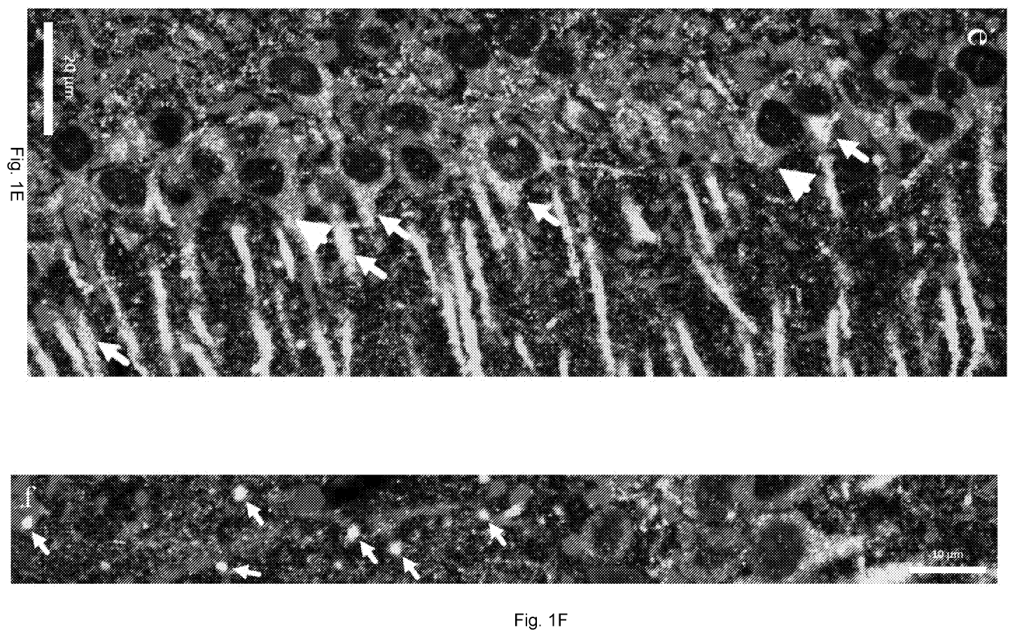

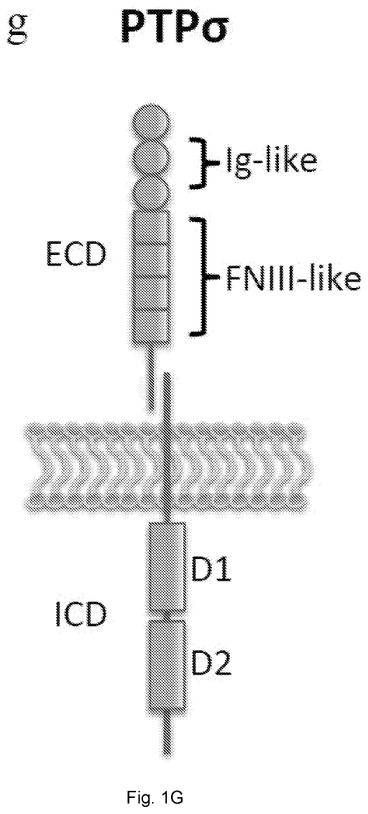

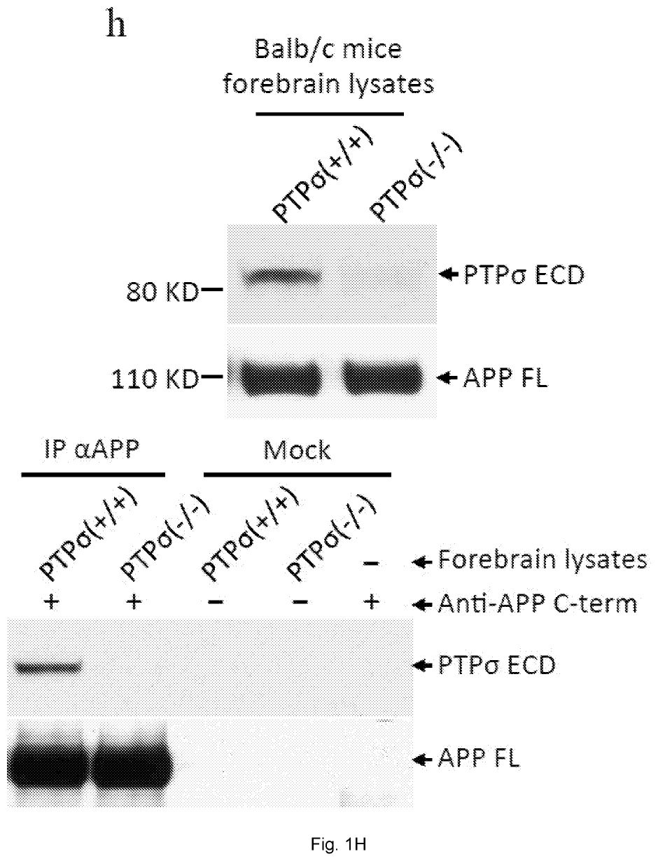

Previously identified as a neuronal receptor of extracellular proteoglycans, PTPG is expressed throughout the adult nervous system, most predominantly in the hippocampus, one of earliest affected brain regions in AD. Using immunohistochemistry and confocal imaging, we found that PTPσ and APP (the precursor of Aβ) colocalize in hippocampal pyramidal neurons of adult rat brains, most intensively in the initial segments of apical dendrites, and in the perinuclear and axonal regions with a punctate pattern (FIG. 1A-F). To assess whether this colocalization reflects a binding interaction between these two molecules, we tested their co-immunoprecipitation from brain homogenates. In brains of rats and mice with different genetic background, using various antibodies of APP and PTPσ, we consistently detected a fraction of PTPσ that co-immunoprecipitates with APP, providing evidence of a molecular complex between these two transmembrane proteins (FIG. 1H, I).

### Genetic Depletion of PTPσ Reduces β-Amyloidogenic Products of APP

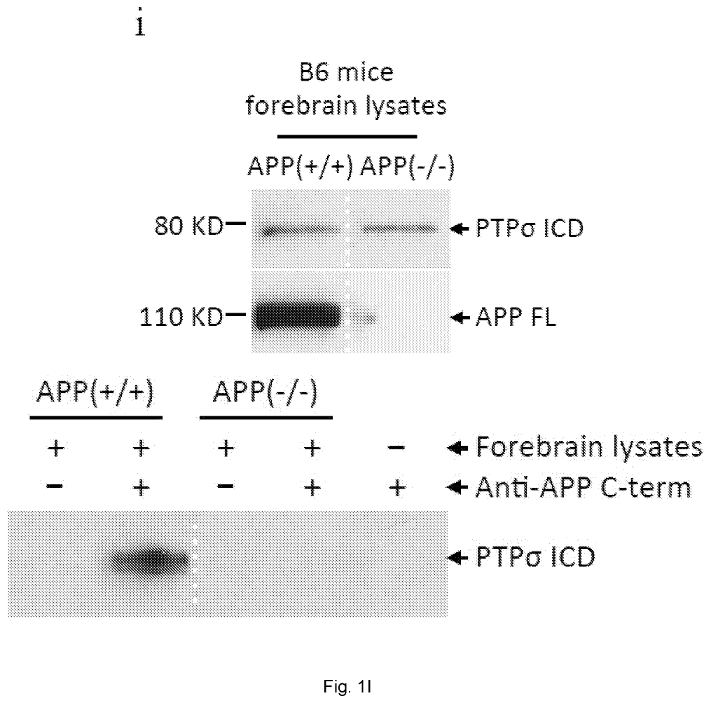

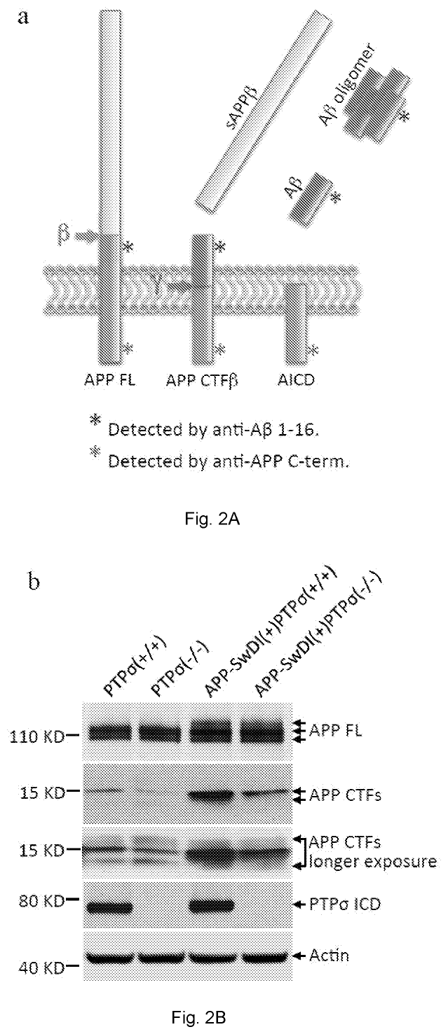

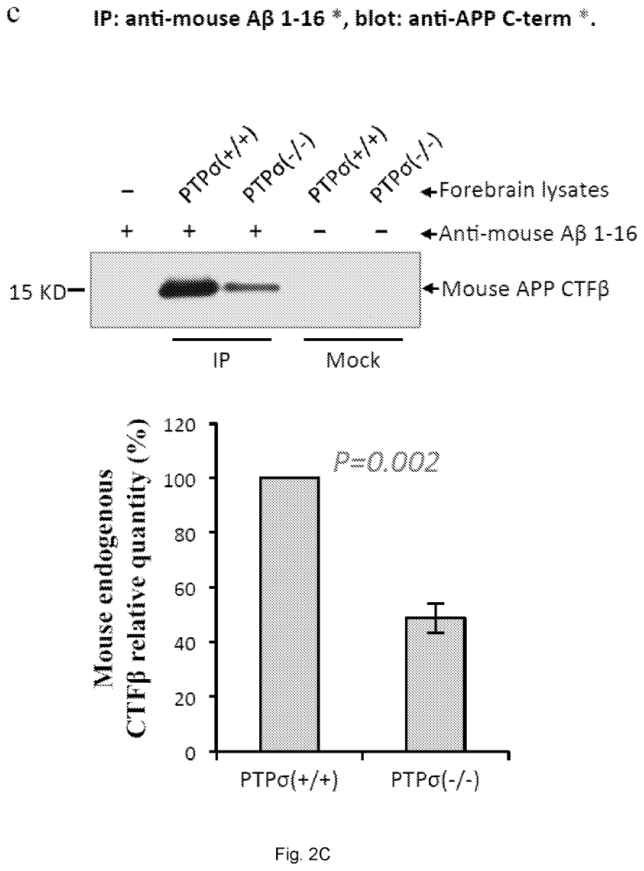

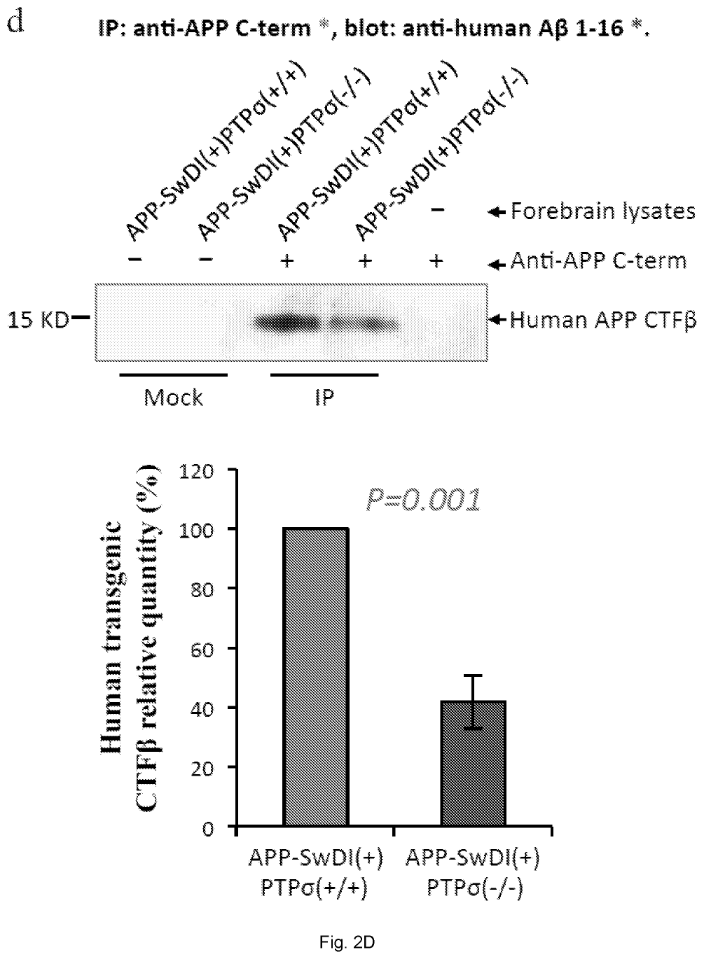

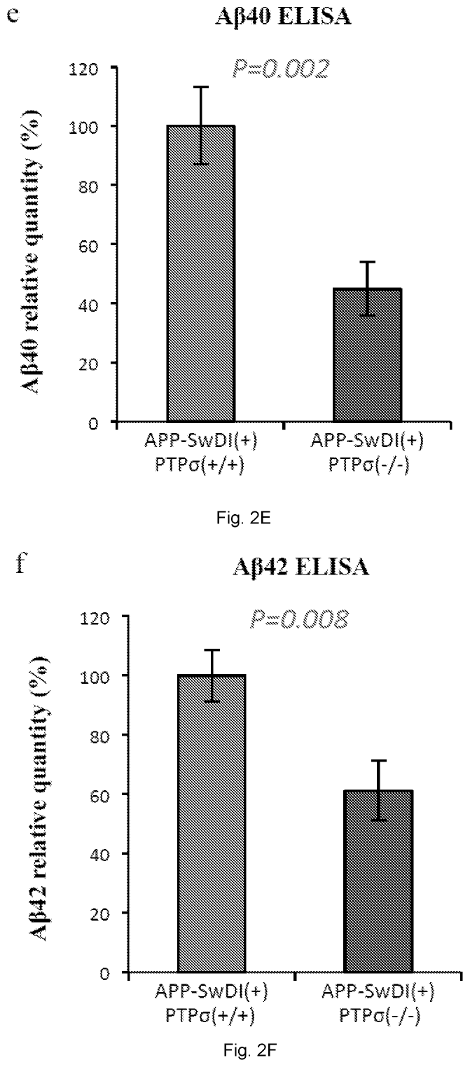

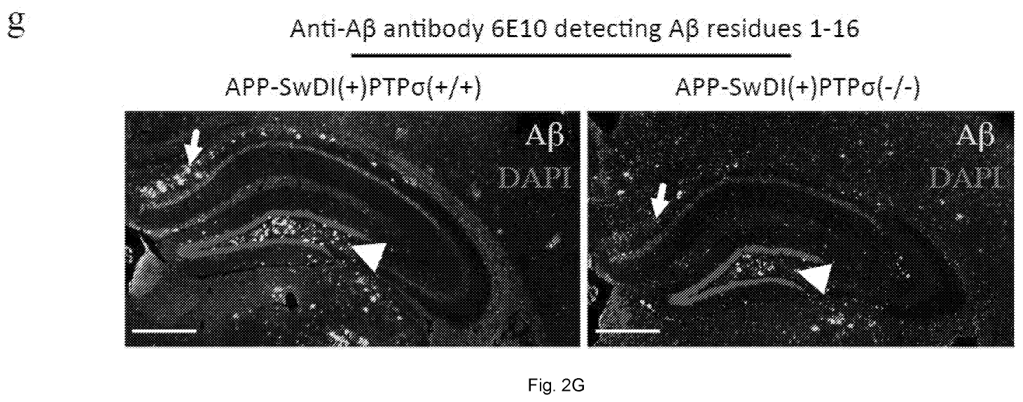

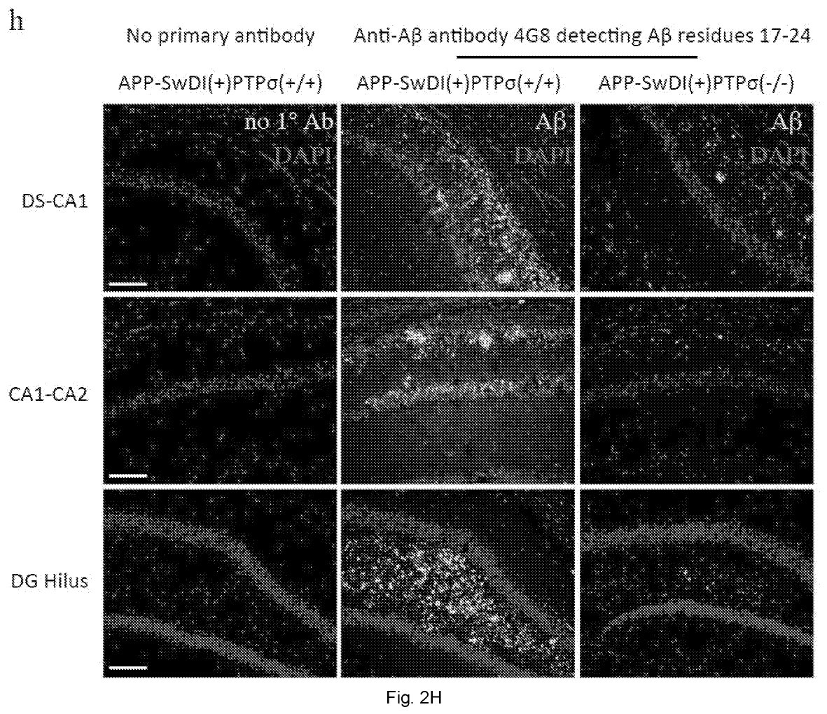

The molecular interaction between PTPσ and APP prompted us to investigate whether PTPσ plays a role in amyloidogenic processing of APP. In neurons, APP is mainly processed through alternative cleavage by either α- or β-secretase. These secretases release the N-terminal portion of APP from its membrane-tethering C-terminal fragment (CTFα or CTFβ, respectively), which can be further processed by the γ-secretase. Sequential cleavage of APP by the β- and γ-secretases is regarded as amyloidogenic processing since it produces Aβ peptides. When overproduced, the Aβ peptides can form soluble oligomers that trigger ramification of cytotoxic cascades, whereas progressive aggregation of Aβ eventually results in the formation of senile plaques in the brains of AD patients (FIG. 2a). To test the effect of PTPσ in this amyloidogenic processing, we analyzed the levels of APP β- and γ-cleavage products in mouse brains with or without PTPσ.

Western blot analysis with protein extracts from mouse brains showed that genetic depletion of PTPσ does not affect the expression level of full length APP (FIG. 2B). However, an antibody against the C-terminus of APP detects a band at a molecular weight consistent with CTFβ, which is reduced in PTPσ-deficient mice as compared to their age-sex-matched wild type littermates (FIG. 2B). Additionally, in two AD mouse models expressing human APP genes with amyloidogenic mutations, we observed a similar decrease of an APP CTF upon PTPσ depletion (FIG. 2B). The TgAPP-SwDI and TgAPP-SwInd mice, each expressing a human APP transgene harboring the Swedish mutation near the β-cleavage site, were crossed with the PTPσ line to generate offsprings that are heterozygous for their respective APP transgene, with or without PTPδ. Because the Swedish mutation carried by these APP transgenes is prone to β-cleavage, the predominant form of APP CTF in these transgenic mice is predicted to be CTFβ. Thus, the reduction of APP CTF in PTPσ-deficient APP transgenic mice may indicate a regulatory role of PTPσ on CTFβ level. However, since the APP C-terminal antibody used in these experiments can recognize both CTFα and CTFβ, as well as the phosphorylated species of these CTFs (longer exposure of western blots showed multiple CTF bands), judging the identity of the reduced CTF simply by its molecular weight may be inadequate. We therefore performed CTFβ immunopurification with subsequent western blot detection, using an antibody that recognizes CTFβ but not CTFα (FIG. 2C, D). With this definitive method, we confirmed that PTPσ depletion decreases the level of CTFβ originated from both mouse endogenous and human transgenic APP.

Because CTFβ is an intermediate proteolytic product between β- and γ-cleavage, its decreased steady state level could result from either reduced production by β-cleavage or increased degradation by subsequent γ-secretase cleavage (FIG. 2A). To distinguish between these two possibilities, we measured the level of Aβ peptides, which are downstream products from CTFβ degradation by γ-cleavage. Using ELISA assays with brain homogenates from the TgAPP-SwDI mice, we found that PTPσ depletion decreases the levels of Aβ peptides to a similar degree as that of CTFβ (FIG. 2E, F). Consistently, as Aβ peptides gradually aggregate into plaques during aging of the transgenic mice, we observed a substantial decrease of cerebral Aβ deposition in APP transgenic PTPσ-deficient mice as compared to the age-matched APP transgenic littermates expressing wild type PTPσ (FIG. 2G, H). Thus, the concurrent decrease of β- and γ-cleavage products argues against an increased γ-secretase activity, but instead suggests a reduced β-secretase cleavage of APP, which suppresses not only the level of CTFβ but also downstream Aβ production in PTPσ-deficient brains.

### Curtailed Progression of β-Amyloidosis in the Absence of PTPσ

Progressive cerebral Aβ aggregation (β-amyloidosis) is regarded as a benchmark of AD progression. To investigate the effects of PTPσ on this pathological development, we monitored Aβ deposits in the brains of 9-month old (mid-aged) and 16-month old (aged) TgAPP-SwDI mice. At age of 9 to 11 months, Aβ deposits are found predominantly in the hippocampus, especially in the hilus of the dentate gyrus (DG) (FIG. 2G, H). By 16 months, the pathology spreads massively throughout the entire brain. The propagation of Aβ deposition, however, is curbed by genetic depletion of PTPσ, as quantified using the DG hilus as a representative area (FIG. 21). Between the ages of 9 and 16 months, the Aβ burden is more than doubled in TgAPP-SwDI mice expressing wild type PTPσ [APP-SwDI(+)PTPσ(+/+)], but only shows marginal increase in the transgenic mice lacking functional PTPσ [APP-SwDI(+)PTPσ(−/−)]. Meanwhile, the Aβ loads measured in 9-month old APP-SwDI(+)PTPσ(+/+) mice are similar to those of 16-month old APP-SwDI(+)PTPσ(−/−) mice (p=0.95), indicating a restraint of disease progression by PTPσ depletion (FIG. 21).

### Decreased BACE1-APP Affinity in PTPσ-Deficient Brains

Consistent with these observations that suggest a facilitating role of PTPσ in APP β-cleavage, our data further reveal that PTPG depletion weakens the interaction of APP with BACE1, the β-secretase in the brain. To test the in vivo affinity between BACE1 and APP, we performed co-immunoprecipitation of the enzyme and substrate from mouse brain homogenates in buffers with serially increased detergent stringency. Whereas BACE1-APP association is nearly equal in wild type and PTPσ-deficient brains under mild buffer conditions, increasing detergent stringency in the buffer unveils that the molecular complex is more vulnerable to dissociation in brains without PTPσ (FIG. 3). Thus a lower BACE1-APP affinity in PTPσ-deficient brains may likely be an underlying mechanism for the decreased levels of CTFβ and its derivative A3.

Although it cannot be ruled out that some alternative uncharacterized pathway may contribute to the parallel decrease of CTFβ and Aβ in PTPσ-deficient brains, these data consistently support the notion that PTPσ regulates APP amyloidogenic processing, likely via facilitation of BACE1 activity on APP, the initial process of Aβ production.

### The Specificity of β-Amyloidogenic Regulation by PTPσ

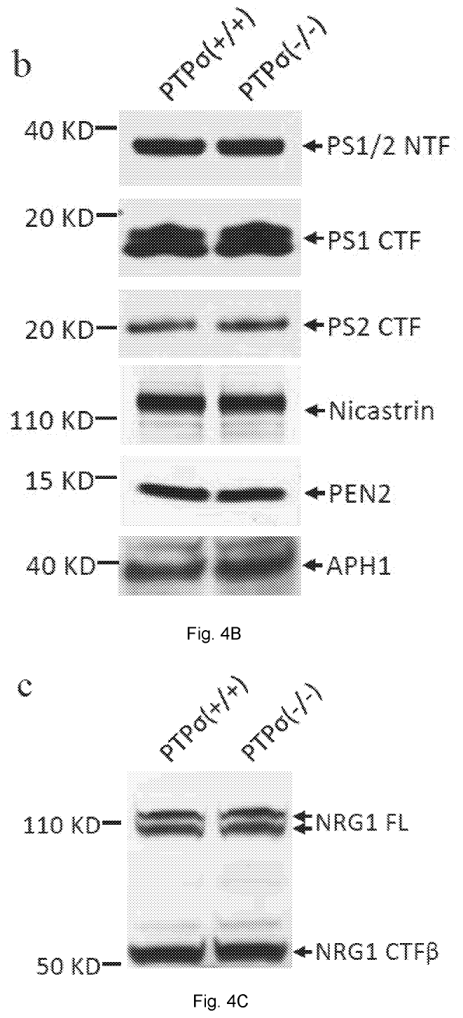

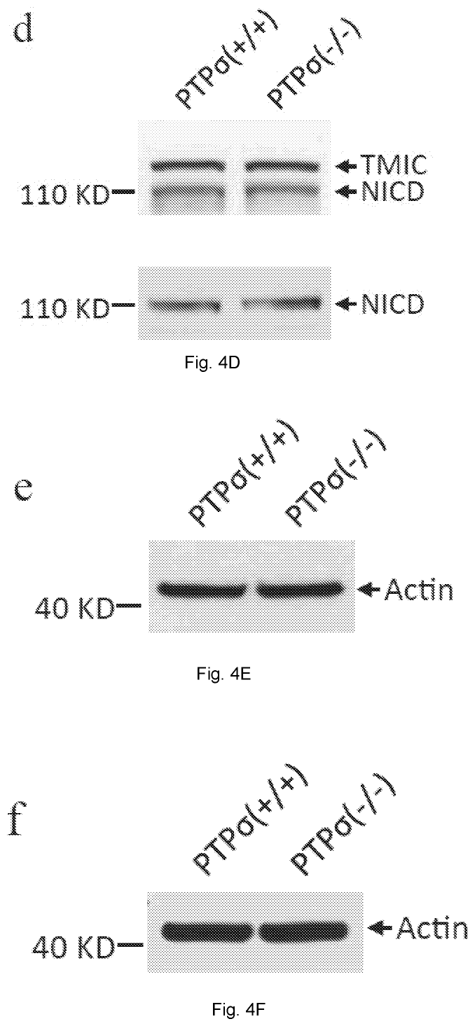

The constraining effect of PTPσ on APP amyloidogenic products led us to further question whether this observation reflects a specific regulation of APP metabolism, or alternatively, a general modulation on the β- and γ-secretases. We first assessed the expression level of these secretases in mouse brains with or without PTPσ, and found no change for BACE1 or the essential subunits of γ-secretase (FIG. 4A, B). Additionally, we tested whether PTPG broadly modulates β- and γ-secretase activities, by examining the proteolytic processing of their other substrates. Besides APP, Neuregulin1 (NRG1) and Notch are the major in vivo substrates of BACE1 and γ-secretase, respectively. Neither BACE1 cleavage of NRG1 nor γ-secretase cleavage of Notch is affected by PTPσ deficiency (FIG. 4C, D). Taken together, these data rule out a generic modulation of β- and γ-secretases, but rather suggest a specificity of APP amyloidogenic regulation by PTPσ.

### PTPσ Depletion Relieves Neuroinflammation and Synaptic Impairment in APP Transgenic Mice

Substantial evidence from earlier studies has established that overproduction of Aβ in the brain elicits multiplex downstream pathological events, including chronic inflammatory responses of the glia, such as persistent astrogliosis. The reactive (inflammatory) glia would then crosstalk with neurons, evoking a vicious feedback loop that amplifies neurodegeneration during disease progression.

The TgAPP-SwDI model is one of the earliest to develop neurodegenerative pathologies and behavioral deficits among many existing AD mouse models. We therefore chose these mice to further examine the role of PTPG in AD pathologies downstream of neurotoxic A3.

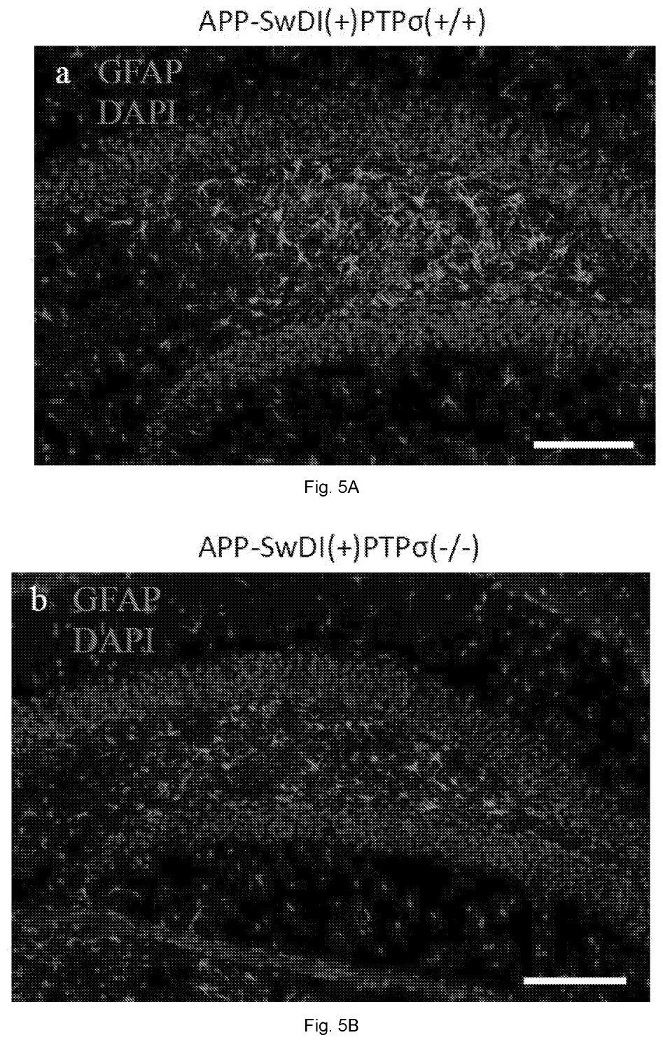

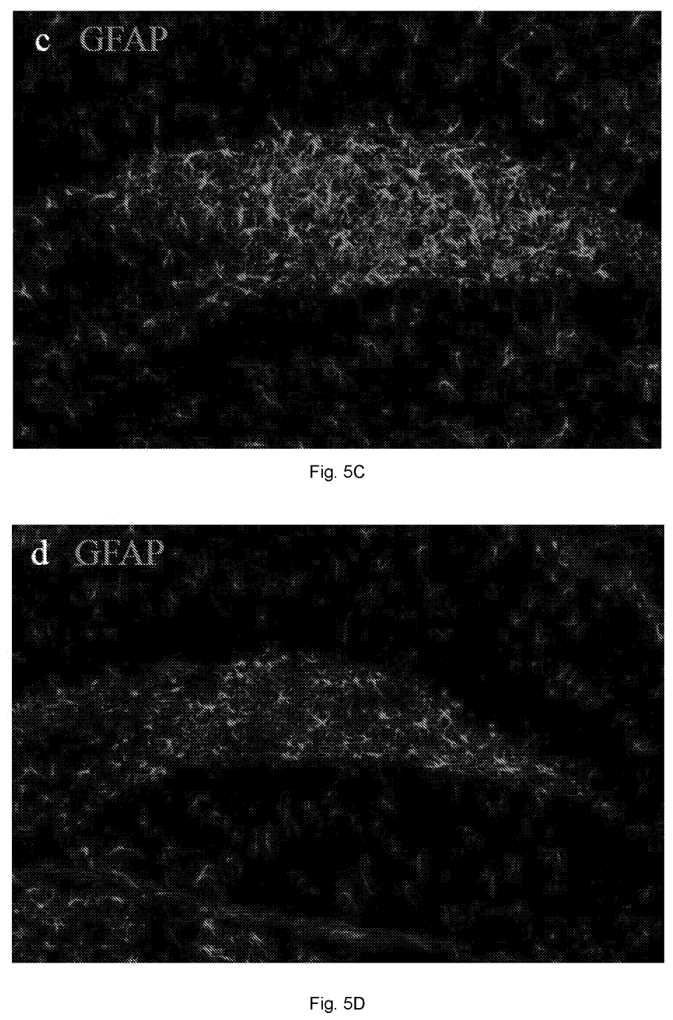

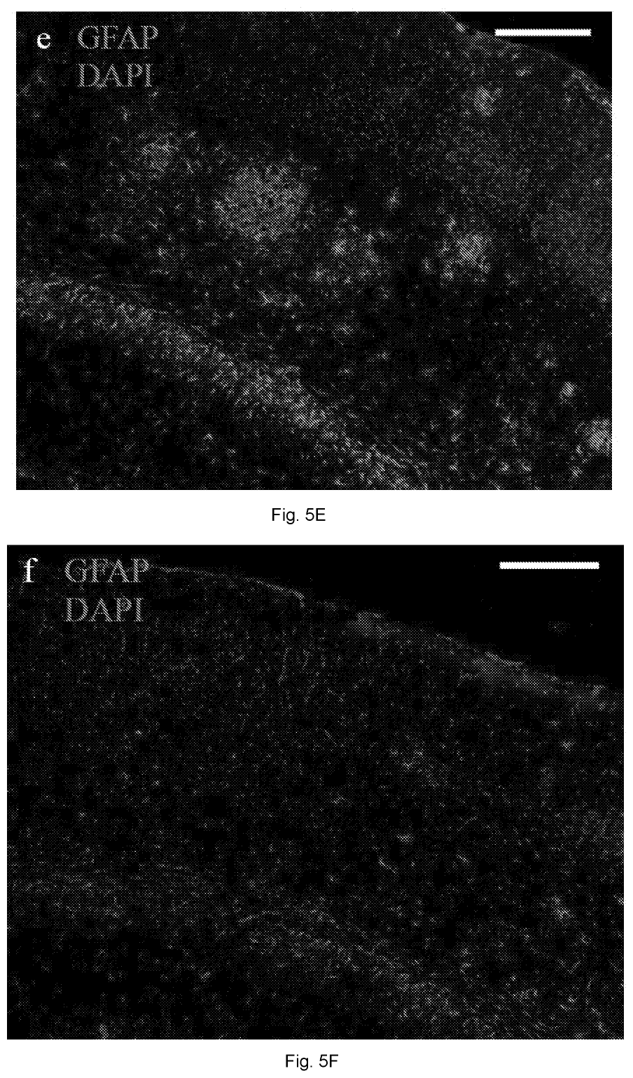

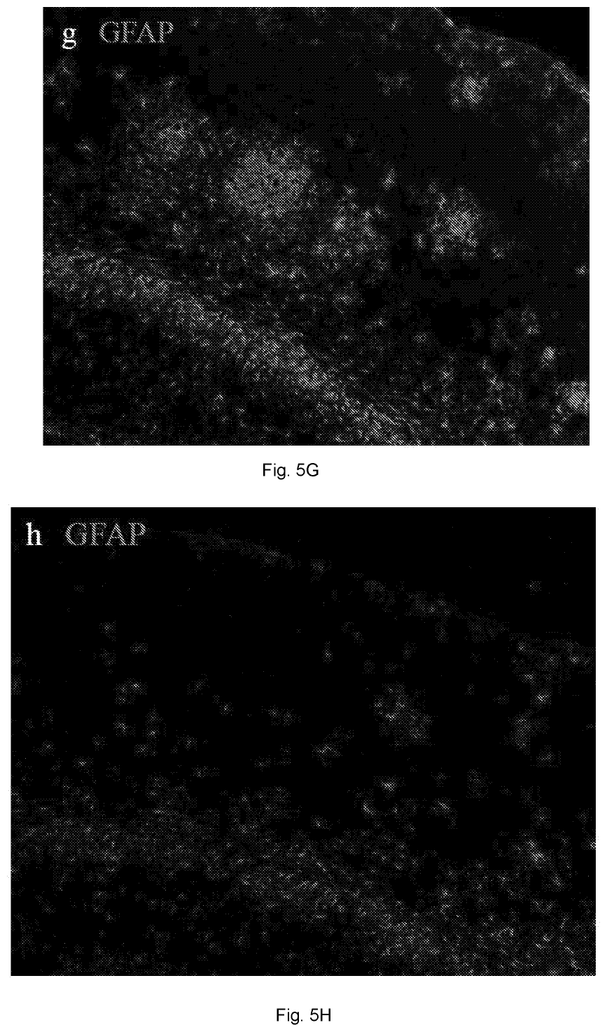

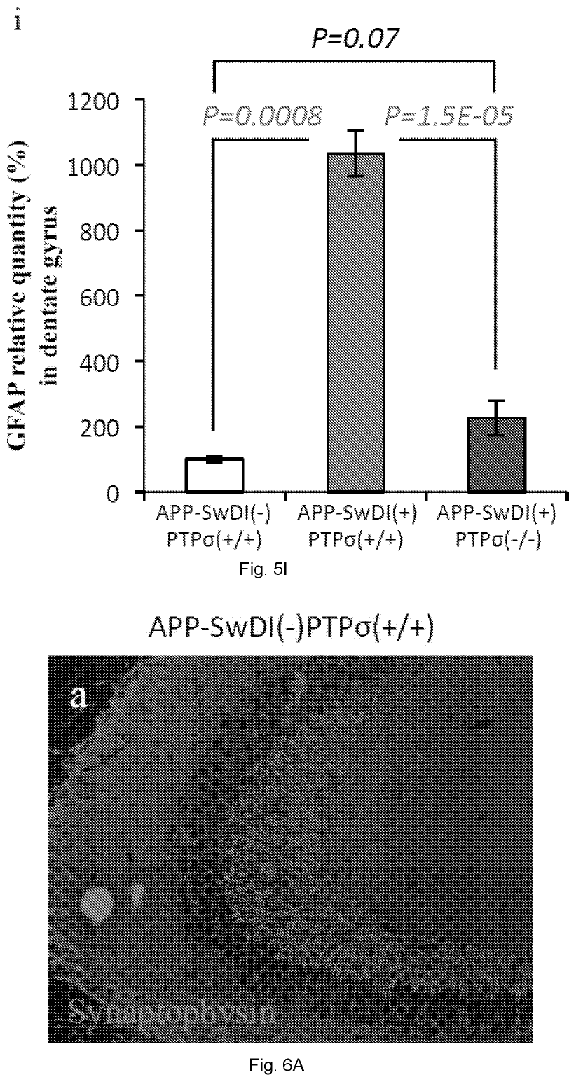

The APP-SwDI(+)PTPσ(+/+) mice, which express the TgAPP-SwDI transgene and wild type PTPσ, have developed severe neuroinflammation in the brain by the age of 9 months, as measured by the level of GFAP (glial fibrillary acidic protein), a marker of astrogliosis (FIG. 5). In the DG hilus, for example, GFAP expression level in the APP-SwDI(+)PTPσ(+/+) mice is more than tenfold compared to that in age-matched non-transgenic littermates [APP-SwDI(−)PTPσ(+/+)]. PTPσ deficiency, however, effectively attenuates astrogliosis induced by the amyloidogenic transgene. In the APP-SwDI(+)PTPσ(−/−) brains, depletion of PTPσ restores GFAP expression in DG hilus back to a level close to that of non-transgenic wild type littermates (FIG. 5k).

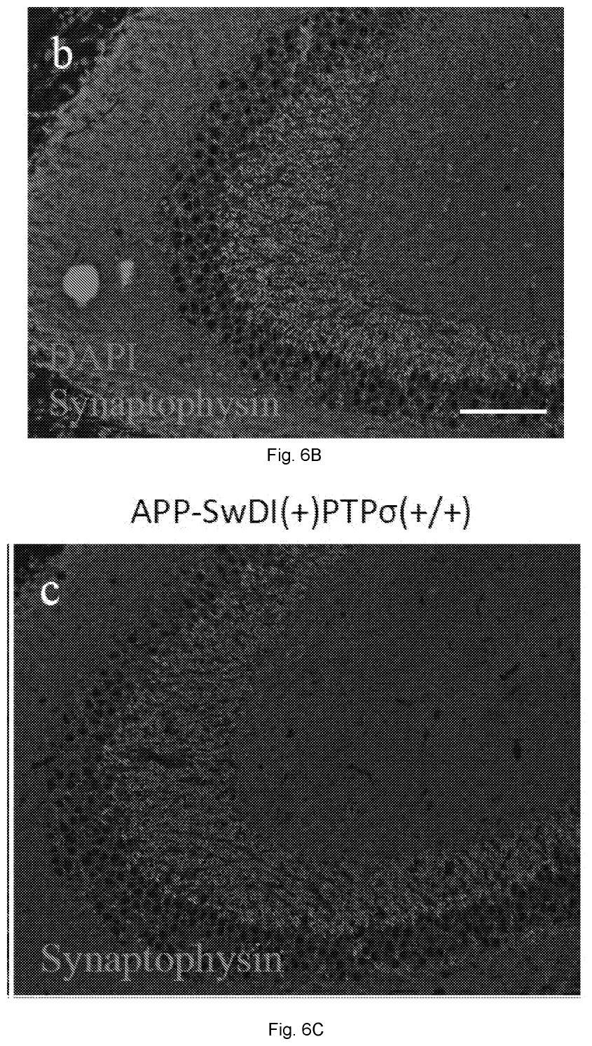

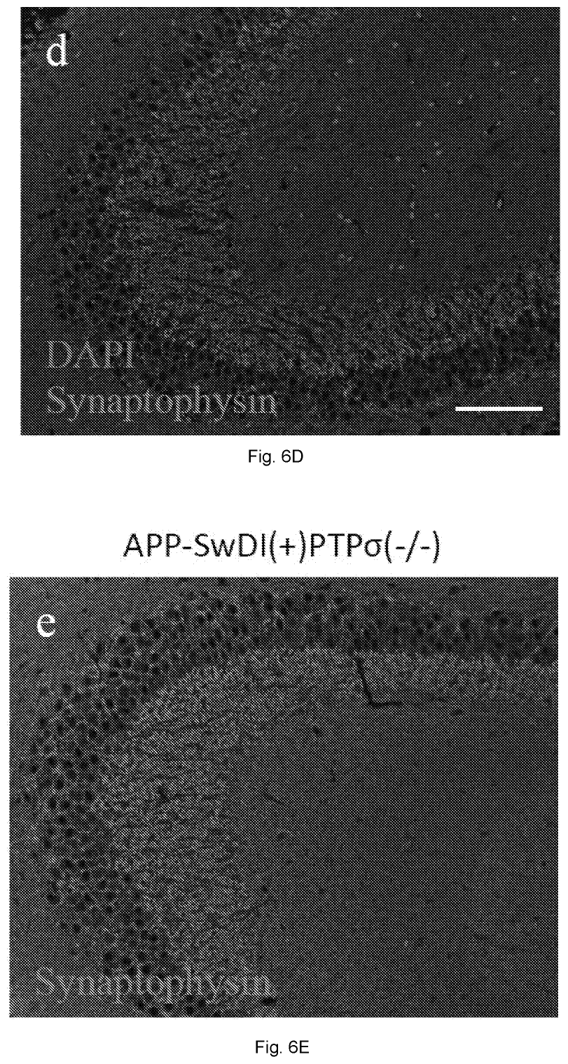

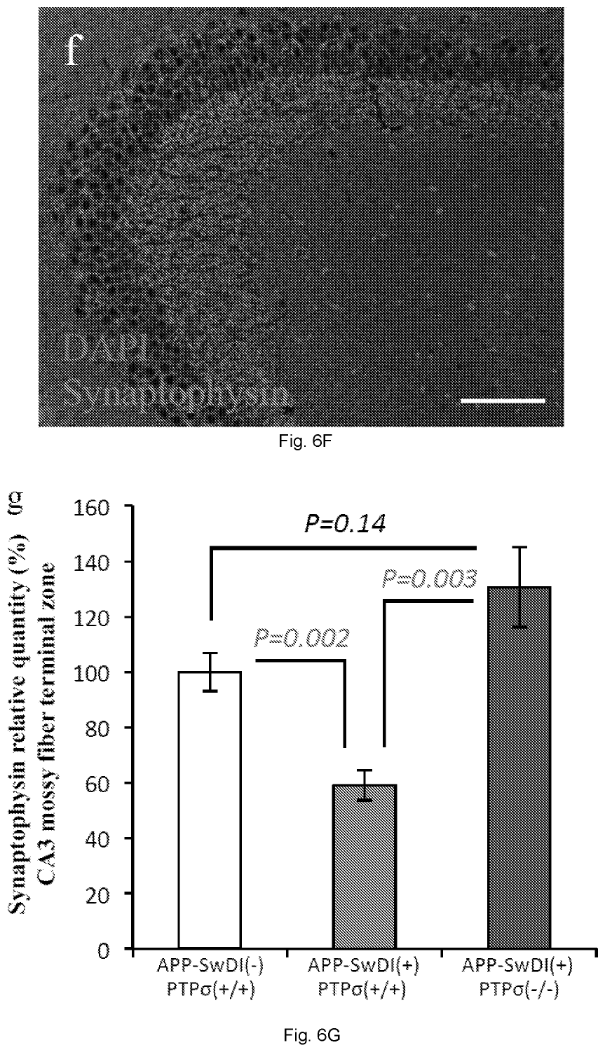

Among all brain regions, the most affected by the expression of TgAPP-SwDI transgene appears to be the hilus of the DG, where Aβ deposition and astrogliosis are both found to be the most severe (FIG. 2G, H; FIG. 5). We therefore questioned whether the pathologies in this area have an impact on the mossy fiber axons of DG pyramidal neurons, which project through the hilus into the CA3 region, where they synapse with the CA3 dendrites. Upon examining the presynaptic markers in CA3 mossy fiber terminal zone, we found decreased levels of Synaptophysin and Synapsin-1 in the APP-SwDI(+)PTPσ(+/+) mice, comparing to their age-matched non-transgenic littermates (FIG. 6, data not shown for Synapsin-1). Such synaptic impairment, evidently resulting from the expression of the APP transgene and possibly the overproduction of Aβ, is reversed by genetic depletion of PTPσ in the APP-SwDI(+)PTPσ(−/−) mice (FIG. 6).

Interestingly, we noticed that the APP-SwDI(+)PTPσ(−/−) mice sometimes express higher levels of presynaptic markers in the CA3 terminal zone than their age-matched non-transgenic wild type littermates (FIG. 6g). This observation, although not statistically significant in our quantification analysis, may suggest an additional synaptic effect of PTPσ that is independent of the APP transgene, as observed in previous studies.

### Tau Pathology in Aging AD Mouse Brains is Dependent on PTPσ

Neurofibrillary tangles composed of hyperphosphorylated and aggregated Tau are commonly found in AD brains. These tangles tend to develop in a hierarchical pattern, appearing first in the entorhinal cortex before spreading to other brain regions. The precise mechanism of tangle formation, however, is poorly understood. The fact that Tau tangles and Aβ deposits can be found in separate locations in postmortem brains has led to the question of whether Tau pathology in AD is independent of Aβ accumulation. Additionally, despite severe cerebral β-amyloidosis in many APP transgenic mouse models, Tau tangles have not been reported, further questioning the relationship between Aβ and Tau pathologies in vivo.

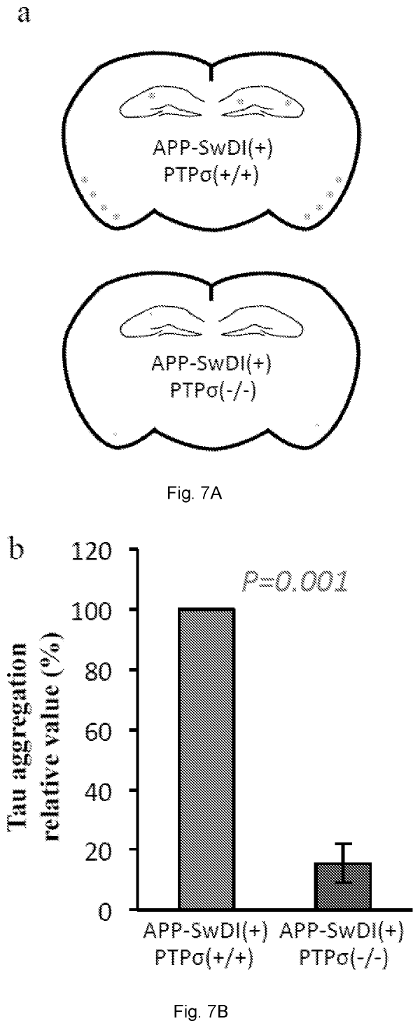

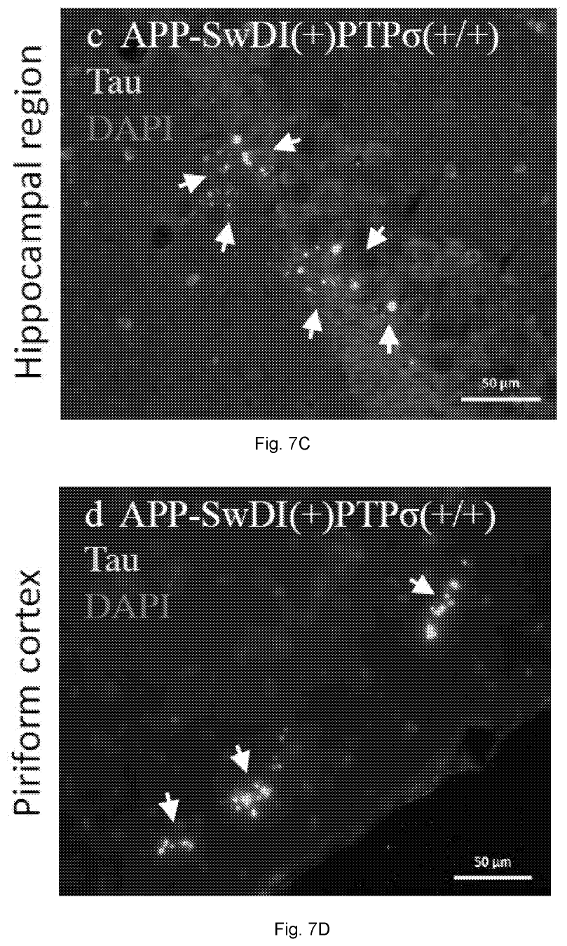

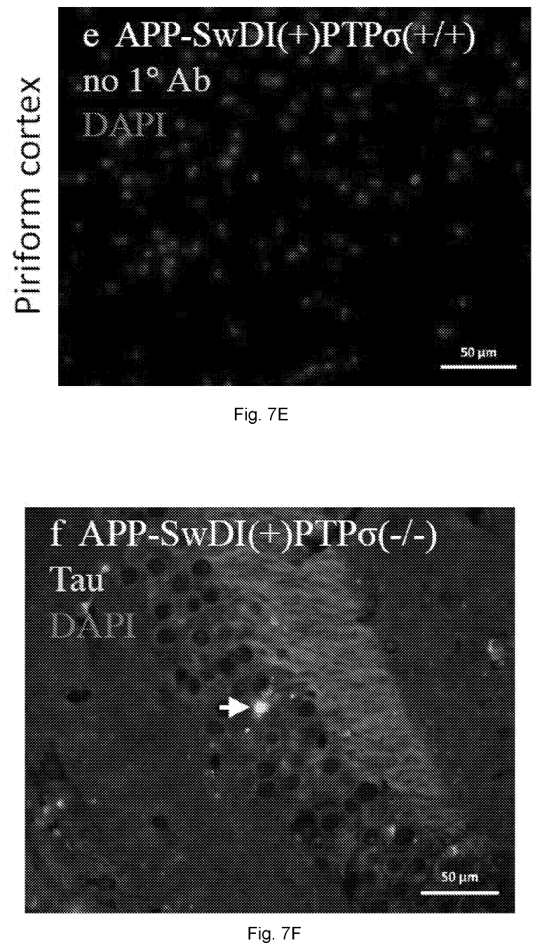

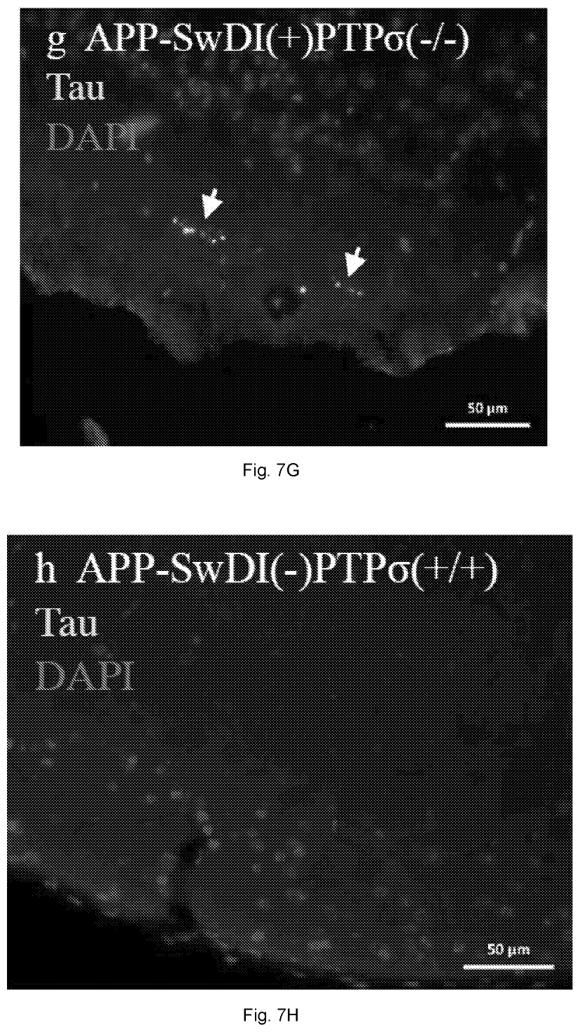

Nonetheless, a few studies did show non-tangle like assemblies of Tau in dystrophic neurites surrounding Aβ plaques in APP transgenic mouse lines, arguing that Aβ can be a causal factor for Tau dysregulation, despite that the precise nature of Tau pathologies may be different between human and mouse. In our histological analysis using an antibody against the proline-rich domain of Tau, we observed Tau aggregation in the brains of both TgAPP-SwDI and TgAPP-SwInd mice during the course of aging (around 9 months for the APP-SwDI(+)PTPσ(+/+) mice and 15 months for the APP-SwInd(+)PTPσ(+/+) mice) (FIG. 7). Such aggregation is not seen in aged-matched non-transgenic littermates (FIG. 7h), suggesting that it is a pathological event downstream from the expression of amyloidogenic APP transgenes, possibly a result of Aβ cytotoxicity. Genetic depletion of PTPσ, which diminishes Aβ levels, suppresses Tau aggregation in both TgAPP-SwDI and TgAPP-SwInd mice (FIG. 7).

In both TgAPP-SwDI and TgAPP-SwInd mice, the Tau aggregates are found predominantly in the molecular layer of the piriform and entorhinal cortices, and occasionally in the hippocampal region (FIG. 7), reminiscent of the early stage tangle locations in AD brains. Upon closer examination, the Tau aggregates are often found in punctate shapes, likely in debris from degenerated cell bodies and neurites, scattered in areas free of nuclear staining. Rarely, a few are in fibrillary structures, probably in degenerated cells before disassembling. To confirm these findings, we used an additional antibody recognizing the C-terminus of Tau and detected the same morphologies as well as distribution pattern (FIG. 7A).

Consistent with the findings in postmortem AD brains, the distribution pattern of Tau aggregates in the TgAPP-SwDI brain does not correlate with that of Aβ deposition, which is pronounced in the hippocampus yet only sporadic in the piriform or entorhinal cortex at the age of 9 months (FIG. 2G, H). Given that the causation of Tau pathology in these mice is possibly related to the overproduced Aβ, the segregation of predominant areas for Aβ and Tau depositions may indicate that the cytotoxicity originates from soluble Aβ instead of the deposited amyloid. It is also evident that neurons in different brain regions are not equally vulnerable to developing Tau pathology.

We next examined whether the expression of APP transgenes or genetic depletion of PTPσ regulates Tau aggregation by changing its expression level and/or phosphorylation status. Western blot analysis of brain homogenates showed that Tau protein expression is not affected by the APP transgenes or PTPσ, suggesting that the aggregation may result from local misfolding of Tau rather than an overexpression of this protein. These experiments with brain homogenates also revealed that TgAPP-SwDI or TgAPP-SwInd transgene, which apparently causes Tau aggregation, does not enhance the phosphorylation of Tau residues including Serine191, Therionine194, and Therionine220 (data not shown), whose homologues in human Tau (Serine202, Therionine205, and Therionine231) are typically hyperphosphorylated in neurofibrillary tangles. These findings are consistent with a recent quantitative study showing similar post-translational modifications of Tau in wild type and TgAPP-SwInd mice. Furthermore, unlike previously reported, we could not detect these phosphorylated residues in the Tau aggregates, suggesting that the epitopes are either missing (residues not phosphorylated or cleaved off) or embedded inside the misfolding. Given the complexity of Tau post-translational modification, one cannot rule out that the aggregation may be mediated by some unidentified modification(s) of Tau. It is also possible that other factors, such as molecules that bind to Tau, may precipitate the aggregation.

Although the underlying mechanism is still unclear, our finding of Tau pathology in these mice establishes a causal link between the expression of amyloidogenic APP transgenes and a dysregulation of Tau assembly. Our data also suggest a possibility that PTPσ depletion may suppress Tau aggregation by reducing amyloidogenic products of APP.

Malfunction of Tau is broadly recognized as a neurodegenerative marker since it indicates microtubule deterioration. The constraining effect on Tau aggregation by genetic depletion of PTPσ thus provides additional evidence for the role of this receptor as a pivotal regulator of neuronal integrity.

### PTPσ Deficiency Rescues Behavioral Deficits in AD Mouse Models

We next assessed whether the alleviation of neuropathologies by PTPσ depletion is accompanied with a rescue from AD relevant behavioral deficits. The most common symptoms of AD include short-term memory loss and apathy among the earliest, followed by spatial disorientation amid impairment of many cognitive functions as the dementia progresses. Using Y maze and novel object assays as surrogate models, we evaluated these cognitive and psychiatric features in the TgAPP-SwDI and TgAPP-SwInd mice.

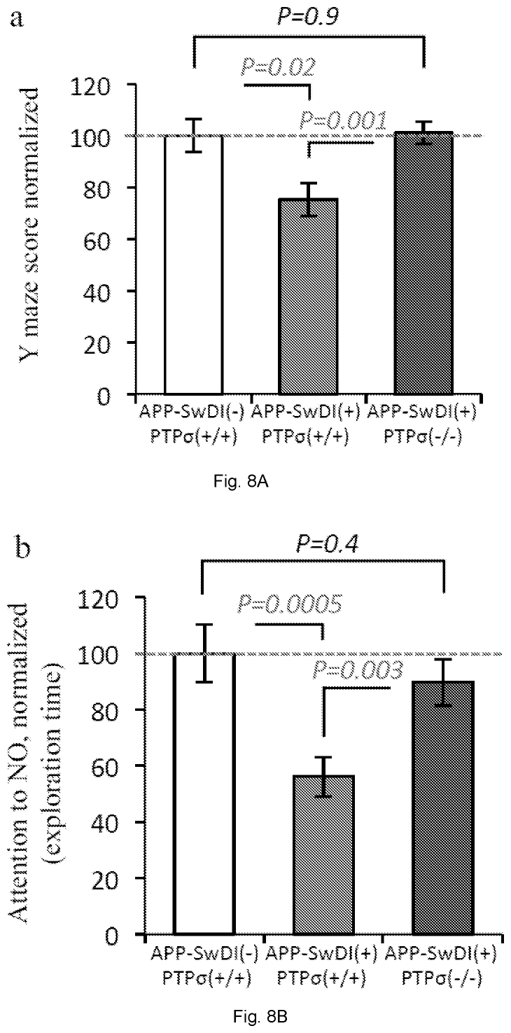

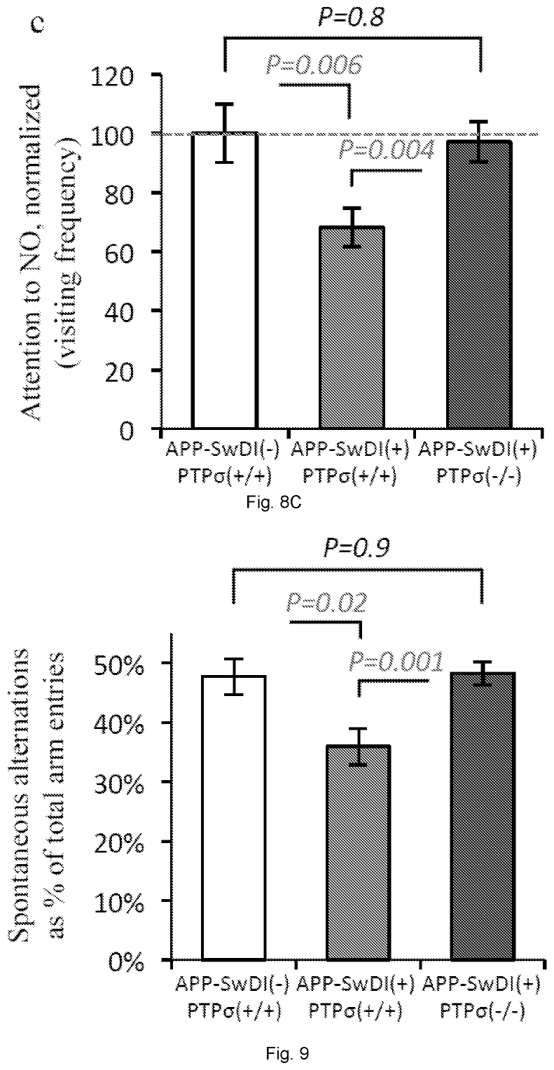

The Y-maze assay, which allows mice to freely explore three identical arms, measures their short-term spatial memory. It is based on the natural tendency of mice to alternate arm exploration without repetitions. The performance is scored by the percentage of spontaneous alternations among total arm entries, and a higher score indicates better spatial navigation. Compared to the non-transgenic wild type mice within the colony, the APP-SwDI(+)PTPσ(+/+) mice show a clear deficit in their performance. Genetic depletion of PTPσ in the APP-SwDI(+)PTPσ(−/−) mice, however, unequivocally restores the cognitive performance back to the level of non-transgenic wild type mice (FIG. 8A, FIG. 9).

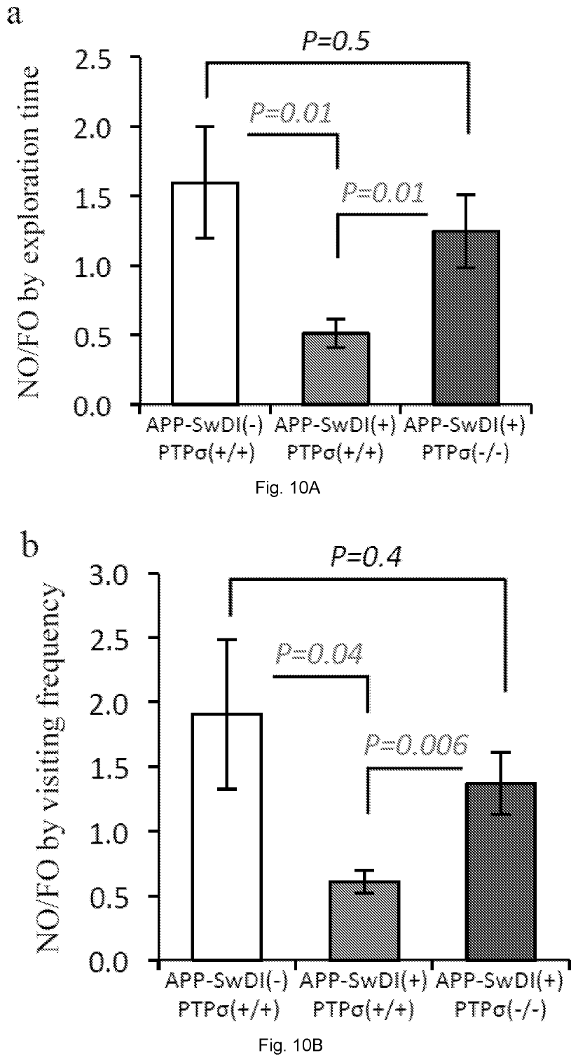

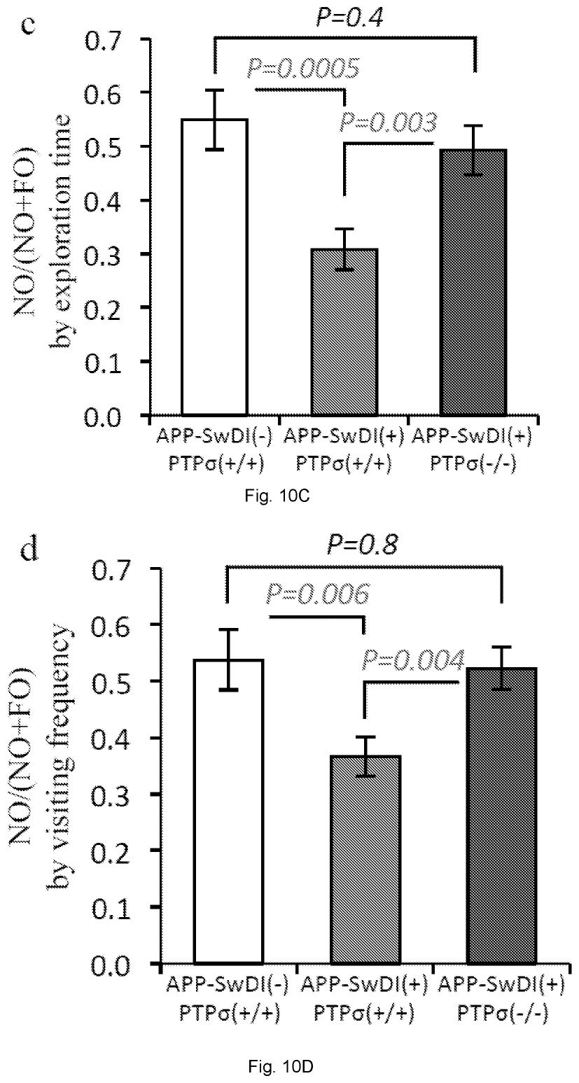

Apathy, the most common neuropsychiatric symptom reported among individuals with AD, is characterized by a loss of motivation and diminished attention to novelty, and has been increasingly adopted into early diagnosis of preclinical and early prodromal AD. Many patients in early stage AD lose attention to novel aspects of their environment despite their ability to identify novel stimuli, suggesting an underlying defect in the circuitry responsible for further processing of the novel information. As a key feature of apathy, such deficits in attention to novelty can be accessed by the “curiosity figures task” or the “oddball task” in patients. These visual-based novelty encoding tasks are very similar to the novel object assay for rodents, which measures the interest of animals in a novel object (NO) when they are exposed simultaneously to a prefamiliarized object (FO). We therefore used this assay to test the attention to novelty in the APP transgenic mice. When mice are pre-trained to recognize the FO, their attention to novelty is then measured by the discrimination index denoted as the ratio of NO exploration to total object exploration (NO+FO), or alternatively, by the ratio of NO exploration to FO exploration. Whereas both ratios are commonly used, a combination of these assessments provides a more comprehensive evaluation of animal behavior. In this test, as indicated by both measurements, the expression of APP-SwDI transgene in the APP-SwDI(+)PTPσ(+/+) mice leads to a substantial decrease in NO exploration as compared to non-transgenic wild type mice (FIG. 8B, C; FIG. 10). Judging by their NO/FO ratios, it is evident that both the transgenic and non-transgenic groups are able to recognize and differentiate between the two objects (FIG. 10A, B). Thus, the reduced NO exploration by the APP-SwDI(+)PTPσ(+/+) mice may reflect a lack of interest in the NO or an inability to shift attention to the NO. Once again, this behavioral deficit is largely reversed by PTPG deficiency in the APP-SwDI(+)PTPσ(−/−) mice (FIG. 8B, C; FIG. 10), consistent with previous observation of increased NO preference in the absence of PTPσ.

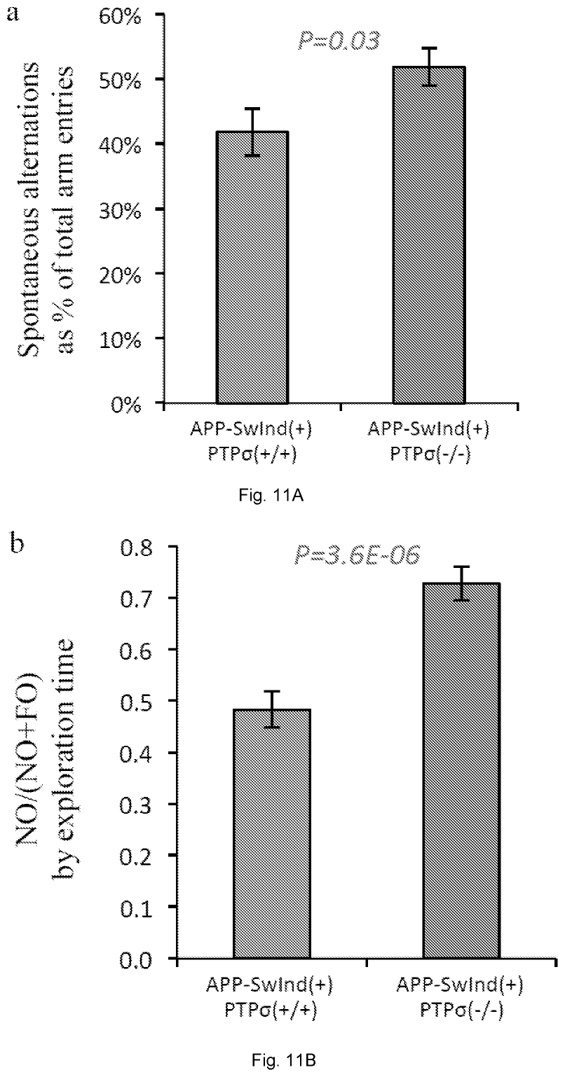

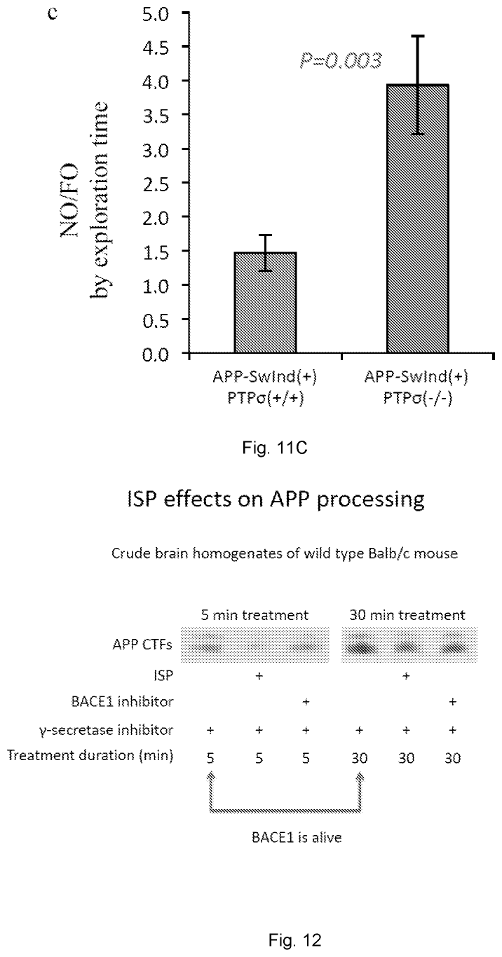

To further verify the effects of PTPG on these behavioral aspects, we additionally tested the TgAPP-SwInd mice in both assays and observed similar results, confirming an improvement on both short-term spatial memory and attention to novelty upon genetic depletion of PTPG (FIG. 11).

FIG. 12 illustrates an immunoblot showing the effects ISP in combination with a γ-secretase inhibitor on APP processing compared to a γ-secretase inhibitor administered alone or a BACE1 inhibitor in combination with a γ-secretase inhibitor. As noted in the figure, ISP in combination with a γ-secretase inhibitor substantially inhibited APP processing compared to a γ-secretase inhibitor administered alone or a BACE1 inhibitor in combination with a γ-secretase inhibitor.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

1-23. (canceled)

24: A method inhibiting and/or reducing β-amyloid accumulation and/or Tau aggregation in a subject in need thereof, the method comprising:
administering to the subject an effective amount of a therapeutic comprising a therapeutic peptide, wherein the therapeutic peptide comprises an amino acid sequence with at least 70% identity to SEQ ID NO: 16.

25: The method of claim 24, wherein the therapeutic peptide comprises an amino acid sequence with at least 85% identity to SEQ ID NO: 16.

26: The method of claim 24, wherein the therapeutic peptide comprises a substitution of an amino acid of at least one of residue 4, 5, 6, 7, 9, 10, 12, or 13 of SEQ ID NO: 16 for another amino acid, wherein the amino acid residue4E is substituted with D or Q, amino acid residue 5R is substituted with H, L or K, amino acid residue 6L is substituted with I, V or M, amino acid residue 7K is substituted with R or H, amino acid residue 9N is substituted with E or D, amino acid residue 10D is substituted with E or N, amino acid residue 12L is substituted with I, V or M, and/or amino acid residue 13K is substituted with R or H.

27: The method of claim 24, wherein the therapeutic peptide comprises an amino acid sequence selected from the group consisting of SEQ ID NOs: 9-13 and 16.

28: The method of claim 24, wherein the therapeutic peptide comprises an amino acid sequence of SEQ ID NO: 16.

29: The method of claim 24, wherein the therapeutic agent includes a transport moiety that is linked to the therapeutic peptide and facilitates uptake of the therapeutic peptide by a cell.

30: The method of claim 29, wherein the cell is a nerve cell.

31: The method of claim 29, wherein the transport moiety is an HIV Tat transport moiety.

32: The method of claim 24, wherein the therapeutic agent is administered systemically to the subject being treated.

33: The method of claim 24, wherein the subject has Alzheimer's disease.

34: A method of treating Alzheimer's disease in a subject in need thereof, the method comprising:
administering to the subject an effective amount of a therapeutic agent comprising a therapeutic peptide, wherein the therapeutic peptide comprises an amino acid sequence with at least 70% identity to SEQ ID NO: 16.

35: The method of claim 34, wherein the therapeutic peptide comprises an amino acid sequence with at least 85% identity to SEQ ID NO: 16.

36: The method of claim 34, wherein the therapeutic peptide comprises a substitution of an amino acid of at least one of residue 4, 5, 6, 7, 9, 10, 12, or 13 of SEQ ID NO: 16 for another amino acid, wherein the amino acid residue4E is substituted with D or Q, amino acid residue 5R is substituted with H, L or K, amino acid residue 6L is substituted with I, V or M, amino acid residue 7K is substituted with R or H, amino acid residue 9N is substituted with E or D, amino acid residue 10D is substituted with E or N, amino acid residue 12L is substituted with I, V or M, and/or amino acid residue 13K is substituted with R or H.

37: The method of claim 34, wherein the therapeutic peptide comprises an amino acid sequence selected from the group consisting of SEQ ID NOs: 9-13 and 16.

38: The method of claim 34, wherein the therapeutic peptide comprises an amino acid sequence of SEQ ID NO: 16.

39: The method of claim 34, wherein the therapeutic agent includes a transport moiety that is linked to the therapeutic peptide and facilitates uptake of the therapeutic peptide by a cell.

40: The method of claim 39, wherein the transport moiety is an HIV Tat transport moiety.

41: The method of claim 34, wherein the therapeutic agent is administered systemically to the subject being treated.

42: The method of claim 34, wherein the therapeutic agent is administered in combination with a secretase inhibitor.

43: The method of claim 42, wherein the secretase inhibitor comprises at least one of a BACE1 inhibitor or a γ-secretase inhibitor.
